(19) United States
(12) Patent Application Publication
(10) Pub. No.: US 2025/0387363 A1
(43) Pub. Date: Dec. 25, 2025

(54) LIQUID EPINEPHRINE PRODRUG FORMULATIONS AND LIQUID EPINEPHRINE FORMULATIONS

(71) Applicant: Insignis Therapeutics, Inc., North Haven, CT (US)

(72) Inventor: Mingbao Zhang, Millwood, NY (US)

(21) Appl. No.: 19/244,724

(22) Filed: Jun. 20, 2025

Related U.S. Application Data
(60) Provisional application No. 63/662,843, filed on Jun. 21, 2024.

(51) Int. Cl. A61K 31/222; A61K 47/10; A61K 47/14; A61K 47/22; A61K 47/28

(52) U.S. Cl. CPC A61K 31/222; A61K 47/10; A61K 47/14; A61K 47/22; A61K 47/28

(57) ABSTRACT

The disclosure provides stable liquid non-aqueous epinephrine and epinephrine prodrug formulations, including non-aqueous liquid dipivefrin HCl formulations. The formulations are suitable for use as oral spray, oral film, liquid-filled capsules or buccal, lingual, or sublingual drop formulation. The oral spray, oral film and liquid-filled capsule can be placed on the tongue, under the tongue (sublingual) or in the buccal space. Preferably, the oral spray is a sublingual spray. The disclosure provides a method for systemic delivery of a therapeutically effective amount of epinephrine to a subject comprising orally administering a non-aqueous liquid formulation of an epinephrine prodrug, such as dipivefrin HCl to the subject. The disclosure also includes a method of treatment of a disease amenable to treatment by in vivo delivery of systemic epinephrine comprising administering the disclosed formulations. The disease can be a respiratory disorder, type I allergic reactions including anaphylaxis and urticaria, or the disease can be cancer or a microbial infection.

### CROSS REFERENCE TO RELATED APPLICATION

This application claims priority to U.S. Provisional Appl. No. 63/662,843, filed Jun. 21, 2024, which is hereby incorporated by reference in its entirety.

### BACKGROUND

This disclosure relates to liquid formulations of epinephrine and epinephrine prodrugs including dipivefrin, a prodrug of epinephrine, and methods of treatment using the disclosed formulations to safely and rapidly deliver systemic epinephrine.

Epinephrine, also known as adrenaline, is a hormone and neurotransmitter naturally produced by both the adrenal glands and certain neurons. It is widely used, in injectable form, to treat anaphylaxis, a severe allergic reaction, asthma attacks, and to re-establish normal cardiac rhythm during cardiac arrest.

Injectable epinephrine has also been shown to be efficacious for preventing and treating cancer. U.S. Pat. No. 5,925,682 discloses that injecting a mammal with an effective amount of epinephrine results in the significant reduction of tumorous growth.

Regular exercise has also been shown to reduce the risk of a wide spectrum of types of cancer and cancer recurrence, including breast cancer, colon and rectal cancer, pancreatic cancer, prostate cancer, endometrial cancer, ovarian cancer, and lung cancer. This protection may be due to the stimulation of epinephrine secretion in response to exercise. Pedersen et al. (Cell Metabolism, 23, 1-9, Mar. 8, 2016) observed that exercise decreases tumor incidence and growth by over 60% across several mouse tumor models through a direct regulation of NK cell mobilization and trafficking in an epinephrine- and IL-6-dependent manner.

The biologic and pharmacologic effects of epinephrine are brought about by its binding to the alpha and beta adrenergic receptors. The distribution of adrenergic receptors on different cells accounts for the multitude of effects of epinephrine. For example, epinephrine binding to α₁ receptors decreases mucosal edema, causes blood vessel constriction, and increases systolic blood pressure. Epinephrine binding to β₁ receptors leads to increase in blood pressure and heart rate and epinephrine binding to β₂ receptors results in decrease in diastolic blood pressure, vasodilation in skeletal vasculature, relaxation of bronchial smooth muscles, and stabilization of mast cells and basophils/inhibition of inflammatory mediators.

Needle-free approaches to deliver epinephrine systemically for anaphylaxis have been attempted, e.g. inhalation, sublingual, and intranasal routes. Epinephrine inhalation has been shown to be ineffective when used in children because of the number of epinephrine inhalations required. The bad taste of inhaled epinephrine also causes most children to be unable to inhale sufficient epinephrine to achieve therapeutic concentration rapidly and significantly. A study in rabbits showed sublingually administered epinephrine to be systemically absorbed in an amount equivalent amount to IM epinephrine. However, the equivalent sublingual dose (40 mg) was about 100-fold higher than the usual IM dose (0.3 mg). The large dose is necessary for sublingual administration due to mucosal enzymatic degradation of the drug by COMT, as well as poor intrinsic mucosal transportation due to the strong vasoconstriction caused by epinephrine itself.

Intranasal delivery of epinephrine for anaphylaxis was disclosed in US patent application US 2015/0005356 A1 and references cited therein. A reversible catechol-O-methyl transferase (COMT) inhibitor and a vasodilator are required to overcome the mucosal enzymatic degradation by COMT and poor intrinsic mucosal transportation due to the strong vasoconstriction caused by epinephrine itself.

The currently approved injectable epinephrine products and intranasal epinephrine formulations are disclosed to have poor stability due to temperature sensitivity. For example, the recommended storage temperature for EpiPen is 68-77° F. (20-25° C.), with excursions permitted to 59-86° F. (15-30° C.). Even under these controlled storage conditions, regulatory shelf-life specifications for epinephrine products allow up to 20% decline in potency necessitating 10% overage due to degradation. The overage could pose serious risk of overdose for patients using freshly manufactured autoinjectors. Conversely, the continuous decline in potency during storage could also reduce epinephrine's effectiveness over time. Furthermore, the degradants formed from epinephrine decomposition during storage can also pose significant safety risks for patients. The poor stability limits the expiration period for injection epinephrine products to about 18 months.

Under real world conditions where at-risk patients need always carry epinephrine autoinjectors regardless of environmental conditions, the products may be exposed to extreme ranges of temperature, for example being left in a hot car where the temperature can reach up to 60° C. (140° F.) in 60 minutes, as well as in winter months where temperatures below 15° C. may damage the injector mechanism. Moreover, since the epinephrine solution contained in the device is composed mostly of water, there is the possibility for it to freeze at 32° F. or below making it impossible to administer the drug.

There remains a need in the art for epinephrine products that are less temperature sensitive, more stable, needle-free, and easier to use than the currently available epinephrine formulations for treatment of various conditions such as anaphylaxis.

Dipivefrin is a dipivaloyl ester prodrug of epinephrine. Dipivefrin is approved for ocular use as a 0.1% ophthalmic solution indicated as initial therapy for the control of intraocular pressure in chronic open-angle glaucoma. Following absorption, dipivefrin is biotransformed into epinephrine in the body.

### SUMMARY

The disclosure provides stable, liquid oral formulations capable of delivering a therapeutically effective amount of epinephrine to a patient via oral administration when administered as an oral spray, oral film, or buccal, lingual, or sublingual drop formulation. The oral spray and oral film can be placed on the tongue, under the tongue (sublingual) or in the buccal space. Preferably, the oral spray is a sublingual spray. The disclosed liquid formulations provide therapeutic plasma epinephrine, including levels comparable to the plasma levels provided by a IM or subcutaneous epinephrine injection. The disclosed liquid formulations provide pharmacodynamic responses (change in blood pressure and pulse rate) comparable to or greater than the pharmacodynamic responses provided by approved epinephrine IM or subcutaneous injection.

In a first aspect the disclosure provides a non-aqueous liquid epinephrine or epinephrine prodrug formulation, comprising (i) epinephrine, an epinephrine prodrug, or a pharmaceutically acceptable salt of either of the foregoing as a pharmaceutical agent, and (ii) a nonaqueous solvent, wherein the formulation is stable for at least 3 months at temperatures from −20° C. to 60° C. The pharmaceutical agent can be epinephrine or an epinephrine prodrug, such as dipivefrin hydrochloride.

The disclosure also provides a non-aqueous liquid epinephrine or epinephrine prodrug formulation comprising (i) epinephrine, an epinephrine prodrug, or a pharmaceutically acceptable salt of either of the foregoing as a pharmaceutical agent, (ii) at least 30% of the formulation composed of propylene glycol, polyethylene glycol, or glycerol, or a combination thereof, (iii) at least 10% of the formulation composed of dehydrated ethyl alcohol, a terpene or a terpenoid, and (iv) optionally a surfactant.

In an embodiment the formulation comprises epinephrine or an epinephrine prodrug (e.g. dipivefrin hydrochloride) as the pharmaceutical agent, at least 30% propylene glycol, at least 10% dehydrated ethyl alcohol, a terpene or a terpenoid, a surfactant, and optionally a sweetener.

In an embodiment the formulation comprises 10 mg/mL to 300 mg/mL dipivefrin HCl, 10% to 70% (w/v) dehydrated ethyl alcohol, 30% to 70% (w/v) propylene glycol, polyethylene glycol, or glycerol, or a combination thereof, and 0.1%-25% (w/v) of a terpene or a terpenoid selected from L-Menthol, Eucalyptol, and Eugenol, a surfactant comprising 0.1 to 5% (w/v) of a surfactant selected from a bile salt, sodium choleate, sucrose laurate, a lipid such as Labrasol®, Peceol®, Gelucire® 44/14, Gelucire® 48/16, Compritol®, Precirol®, Capryol®90, Lauroglycol™90, a caprylocaproyl macrogol-8 glyceride, a caprylocaproyl polyoxyl-8 glyceride, or a polyoxylglyceride, or a combination of the foregoing, and optionally a sweetener or flavorant or coloring agent.

The disclosure provides methods of delivering a therapeutically effective amount of epinephrine to a subject, comprising orally administering a formulation of the disclosure to the subject. The formulation can be a epinephrine prodrug formulation, such as a dipivefrin hydrochloride, that provides a therapeutically effective plasma epinephrine level following administration. The formulation can be administered by a variety of known pharmaceutical oral administration routes. To achieve rapid therapeutic epinephrine plasma levels a route of oral administration in which epinephrine or the epinephrine prodrug is absorbed through the oral mucosa is advantageous. For example, the method includes administration of the disclosed formulations as an oral spray, liquid filled capsule, oral film, or buccal, lingual, or sublingual drop formulation. The oral spray, oral film, or liquid filled capsule can be placed on the tongue, under the tongue (sublingual) or in the buccal space. Preferably, the oral spray is a sublingual spray.

The disclosure provides method for treating a condition responsive to epinephrine in a subject comprising orally administering a therapeutically effective amount of a formulation disclosed herein to the subject.

The condition responsive to epinephrine can be a Type I allergic reaction, also called a Type I hypersensitivity reaction, which may result from insect stings or bites, foods, drugs, sera, diagnostic testing substances and other allergens, as well as idiopathic anaphylaxis or exercise-induced anaphylaxis. Type I allergic reactions can include any of the following conditions anaphylaxis, allergic asthma, allergic conjunctivitis, allergic rhinitis, anaphylaxis, angioedema, urticaria, eosinophilia. A type I allergic reaction is an immediate reaction involving immunoglobulin E (IgE) mediated release of antibodies against an antigen.

The condition responsive to epinephrine can be a breathing difficulty, such as, asthma, bronchitis, emphysema, croup, or a respiratory infection.

The condition responsive to epinephrine can also be cancer. While epinephrine is not administered alone for the treatment of cancer, it is known to enhance the penetration and activity of some chemotherapeutic agents and is useful as an adjunctive anticancer treatment. For example, the condition responsive to epinephrine can be skin cancer, e.g., melanoma. The disclosure includes a method of administering a formulation of the disclosure as an adjunctive anticancer treatment in combination with at least one additional anticancer treatment, to a subject who has cancer.

The condition responsive to epinephrine can also be a microbial infection, such as a bacterial, viral, fungal, or parasitic infection. The disclosure includes methods of treating a microbial infection in a subject comprising administering a formulation of the disclosure in combination with at least one additional antimicrobial agent.

### BRIEF DESCRIPTION OF THE DRAWINGS

The following is a brief description of the drawings which are presented for the purposes of illustrating the exemplary embodiments disclosed herein and not for the purposes of limiting the same.

FIG. 1 is a graph of mean plasma epinephrine (epi) concentration as a function of time in adult human subjects (N=3) after liquid oral administration of dipivefrin hydrochloride as a sublingual drop, sublingual spray, or lingual spray. Data was obtained in Clinical Study 1, discussed in Example 6.

FIG. 2. Epinephrine Mean Concentration-Time profiles from Example 7 study. A graph of mean plasma epinephrine concentration as a function of time following sublingual spray administration of dipivefrin HCl.

FIG. 3. The epinephrine mean concentration-time profile from Example 8 study. A graph of mean plasma epinephrine concentration as a function of time following IM administration of epinephrine in 5 study subjects.

FIG. 4. The epinephrine mean concentration-time profile from the Example 9 study. Study conditions were the same as Ex. 8 but included more study subjects (N=8).

FIG. 5. A cross study comparison between the epinephrine mean concentration-Time profiles from Clinical Ex. 7 and Epipen Examples 8 and 9. Study period I and study period II refer to the two test periods described in example 7 which are separated by a 1 week washout period. The comparison shows that sublingual dipivefrin HCl liquid dosage forms containing 5 or 10 mg dipivefrin HCl provides a plasma level of epinephrine with a Cₘₐₓ and Tₘₐₓ comparable to that of a 0.3 mg IM injection.

FIG. 6. A cross study comparison between the median SBP (Systolic Blood Pressure) Change from Baseline-Time profiles from Clinical Ex. 7 and Epipen Example 8. The comparison shows that sublingual dipivefrin HCl liquid dosage form containing 10 mg dipivefrin HCl provides a greater SBP response compared to EpiPen 0.3 mg injection two hours postdose.

FIG. 7. A cross study comparison between the median DBP (Diastolic Blood Pressure) Change from Baseline-Time profiles from Clinical Ex. 7 and Epipen Example 8. The comparison shows that sublingual dipivefrin HCl liquid dosage form containing 10 mg dipivefrin HCl provides a greater DBP response compared to EpiPen 0.3 mg injection two hours postdose.

FIG. 8. A cross study comparison between the median PR (Pulse Rate) Change from Baseline-Time profiles from Clinical Ex. 7 and Epipen Example 8. The comparison shows that sublingual dipivefrin HCl liquid dosage form containing 10 mg dipivefrin HCl provides a comparable or greater PR response compared to EpiPen 0.3 mg injection two hours postdose.

### DETAILED DESCRIPTION

### Terminology

The terminology used herein is for the purpose of describing embodiments only and is not intended to be limiting. As used herein, the singular forms “a,” “an,” and “the” are intended to include the plural forms, including “at least one,” unless the content clearly indicates otherwise. As used herein, the term “and/or” includes all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

The terms “administer,” “administering,” “administered,” or “administration” refer to any manner of providing a pharmaceutical agent (such as dipivefrin or a pharmaceutically acceptable salt thereof) to a subject or patient. Routes of administration include all routes known by those skilled in the art. Such means include oral, buccal, intravenous, subcutaneous, intramuscular, transdermal, and inhalation, sublingual, intranasal, or topical. Oral administration in which the pharmaceutical agent is predominantly absorbed through the oral mucosa rather than through the GI tract is a preferred route of administration for the liquid epinephrine and epinephrine prodrug (e.g., dipivefrin formulations of this disclosure.) Administration via a liquid oral spray, oral film, or buccal, lingual, or sublingual drop formulation are appropriate routes of administration for the formulations of this disclosure.

“Alkyl” is a straight or branched saturated hydrocarbon radical having the indicated number of carbon atoms, for example 1-8, 1-6, or 1-4 carbon atoms. C₀alkyl is used to indicate a single bond. C₀-C₄alkyl is a single bond or an alkyl group having up to 4 carbon atoms. “Alkyl” includes, for example, methyl, ethyl, n-propyl, isopropyl, n-butyl, sec-butyl, isobutyl, tert-butyl, n-pentyl, iso-pentyl, n-hexyl. A “C₁-C₁₆alkylcarbonyl” is an alkyl group as defined herein, having up to 16 carbon atoms, and bound to the group it substitutes via a carbonyl (C═O) group; an “mono or di-C₁-C₁₆alkylaminocarbonyl” is bound to the group it substitutes via a —NH(C═O)— group; “(C₀-C₁₆alkyl)sulfate” is a directly bound sulfate group, e.g. SO₂, or an alkyl bound to the group it substitutes via a sulfate (e.g. —SO₂—) group.

“Alkoxy” is an alkyl group as defined above with the indicated number of carbon atoms attached through an oxygen bridge. Examples of alkoxy include, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy.

“Cycloalkyl” is a saturated hydrocarbon ring groups, having the specified number of carbon atoms, usually from 3 to 7 carbon atoms. Examples of cycloalkyl groups include cyclopropyl, cyclobutyl, cyclopentyl, or cyclohexyl as well as bridged or caged saturated ring groups such as norborane or adamantane. In the term “(cycloalkyl)alkyl,” cycloalkyl and alkyl are as defined above, and the point of attachment in on the alkyl group.

As used herein, the term “pharmaceutically acceptable salt” is a salt formed from, for example, an acid and a basic group of a dipivefrin composition. Illustrative salts include, but are not limited to, sulfate, citrate, acetate, oxalate, chloride, acid chloride, bromide, iodide, nitrate, phosphate, acid phosphate, isonicotinate, lactate, salicylate, acid citrate, tartrate, oleate, tannnate, pantothenate, bitartrate, ascorbate, succinate, maleate, besylate, gentisinante, fumarate, gluconate, glucaronate, saccharate, formate, benzoate, glutamate, methanesulfonate, ethanesulfonate, benzenesulfonate, p-toluenesulfonate, and pamoate (e.g., 1,1′-methylene-bis-(2-hydroxy-3-naphthoate)) salts. In an embodiment, the salt of dipivefrin is a hydrochloride salt. Unless clearly contraindicated by the context, “dipivefrin” includes the pharmaceutically acceptable salts of dipivefrin.

The term “pharmaceutically acceptable salt” also refers to a salt prepared from a composition having an acidic functional group, such as a carboxylic acid functional group, and a pharmaceutical acceptable inorganic or organic base or a salt prepared from a composition having a basic functional group, such as an amino functional group, and a pharmaceutically acceptable inorganic or organic acid.

A “pharmaceutical agent” means a compound (including for example, dipivefrin), element, or mixture that when administered to a subject, alone or in combination with another compound, element, or mixture, confers, directly or indirectly, a physiological effect on the subject. The indirect physiological effect may occur via a metabolite or other indirect mechanism.

A “dosage form” means a unit of administration of a pharmaceutical agent. Examples of dosage forms include tablets, capsules, oral thin films, orally dissolving (or disintegrating) dosage forms, sprinkles, powders, injections, suspensions, liquids, emulsions, creams, ointments, suppositories, inhalable forms, transdermal forms, intranasal spray (both liquid and powder spray), and the like. A dosage form of a liquid formulation can be a volume that supplies a particular milligram amount of a pharmaceutical agent, for example a 5 ml dosage form containing 5 mg of the pharmaceutical agent. A liquid pharmaceutical dosage form can also be specified by both concentration and volume, e.g. 100 μl of a 50 mg/mL formulation. A liquid dosage form can be formulated for administration as drops, sprays (oral, intranasal, or topical sprays) and in the form of liquid-filled capsules or liquid capsules. Liquid-filled capsules that are easily ruptured in the mouth can be particularly useful to deliver the liquid epinephrine and epinephrine prodrug formulations of this disclosure.

“Drug absorption” or “absorption” typically refers to the process of movement of drug from site of administration of a drug across a barrier into a blood vessel or the site of action, e.g., a drug moving from the gastrointestinal tract into the portal vein or lymphatic system and a drug diffusing into the bloodstream through tissues under the tongue or through the oral mucosa.

“Pharmaceutical compositions” and “pharmaceutical formulations” are compositions comprising at least one pharmaceutical agent, e.g., dipivefrin, and at least one other substance, such as a carrier, excipient, or diluent. Pharmaceutical compositions meet the U.S. FDA's GMP (good manufacturing practice) standards for human or non-human drugs.

The term “carrier” applied to pharmaceutical compositions described herein refers to a diluent, excipient, or vehicle with which a pharmaceutical agent is provided.

A “pharmaceutically acceptable excipient” means an excipient that is useful in preparing a pharmaceutical composition that is safe, non-toxic, and neither biologically nor otherwise undesirable, and includes an excipient that is acceptable for veterinary use as well as human pharmaceutical use. A “pharmaceutically acceptable excipient” as used in the present application includes both one and more than one such excipient.

A “patient” is a human or non-human animal in need of medical treatment. Medical treatment can include treatment of an existing condition, such as a disease or disorder, prophylactic or preventative treatment, or diagnostic treatment. In some embodiments the patient is a human patient. The patient may also be a livestock animal (e.g., sheep, pigs, horses, cows) or a companion animal (dog, cat).

The “epinephrine serum concentration” or “epinephrine plasma concentration” describes the concentration of epinephrine in the blood serum or blood plasma concentration, typically measured in mg, μg, ng, or pg/ml.

“Pharmacodynamics” refers to the factors which determine the biologic response observed relative to the concentration of drug at a site of action of pharmaceutical agent per mL, dL, or L of blood serum, absorbed into the bloodstream after administration. As used herein, measurable plasma concentrations are typically measured in pg/ml, ng/ml, or μg/ml.

“Pharmacokinetics” refers to the factors which determine the attainment and maintenance of the appropriate concentration of drug at a site of action.

The term “subject” includes any human or non-human animal. For example, the methods and compositions disclosed herein can be used to deliver systemic epinephrine to a subject in need thereof. In a particular embodiment, the subject is a human. The subject may also be a livestock animal (e.g., sheep, pigs, horses, cows) or a companion animal (dog, cat).

A “therapeutically effective amount” or “effective amount” is that amount of a pharmaceutical agent to achieve a pharmacological effect or provide a discernible patient benefit. The term “therapeutically effective amount” includes, for example, a prophylactically effective amount—an amount effective to significantly reduce the probability of occurrence of a disorder in a patient at risk for the disorder. An “effective amount” of epinephrine or an epinephrine prodrug (e.g. dipivefrin) is an amount needed to achieve a desired pharmacologic effect or therapeutic improvement without undue adverse side effects. The effective amount of epinephrine or epinephrine prodrug will be selected by those skilled in the art depending on the patient and the type of conditions being treated. It is understood that “an effective amount” or “a therapeutically effective amount” can vary from patient to patient, due to variation in general condition of the subject, the condition being treated, the severity of the condition being treated, and the judgment of the prescribing physician. When discussing a method of treating cancerous tissue, an effective amount includes an amount effective to have a statistically significant and favorable effect on the rate of the patient's cancer cells proliferation over time or on a level of biological marker for the cancer.

The terms “treating” and “treatment” mean implementation of therapy with the intention of reducing in severity or frequency symptoms, elimination of symptoms or underlying cause, prevention of the occurrence of symptoms or their underlying cause, or the improvement or remediation of damage due to a disorder or disease. In certain embodiments “treatment” includes prophylactic treatment, for example, administering an amount of epinephrine or epinephrine prodrug effective to significantly reduce probability of an acute allergic response or reduce the chance of infection by a microbial pathogen a subject exposed or at risk of exposure to an allergen or pathogen. In certain embodiments treatment includes inhibiting the onset of anaphylaxis or reducing the severity of allergy symptoms in a subject exposed to an allergen. In certain embodiments treatment includes inhibiting the onset of pruritus or relieving pruritus symptoms.

### Trademarks

Trademarks are frequently used in the pharmaceutical formulation arts to describe excipients that are well-known and in wide use in the industry. The following list of trademarks with their non-trademarked descriptions may be helpful in reading the description, examples, and claims.
AVICEL—microcrystalline cellulose, (AVICEL PH-101, CAS Reg. No. 9004-34-6 has a 50 μm mean particle size.)
AZONE—laurocapram
CAPRYOL 90—USP NF name: propylene glycol monocaprylate NF, consists of propylene glycol esters of caprylic acid (C8), mainly composed of monoesters and a small fraction of diesters
COMPRITOL—USP NF name: glyceryl dibehenate
DI-PAC—compressible sugar, CAS Reg. No. 57-50-1
GELUCIRE 44/14—USP NF name: Lauroyl Polyoxyl-32 glycerides, FDA name: Lauroyl PEG-32 glycerides
GELUCIRE 48/16—Polyoxyl-32 stearate (type I) NF
LABRASOL—a non-ionic surfactant containing mono-, di- and triglycerides and mono- and di-fatty acid esters of polyethylene glycol, PEG-8 caprylic/capric glycerides, USP NF name: Caprylocaproyl Polyoxyl-8-glycerides, EP Name: Caprylocaproylmacrogol-8 glycerides
LAUROGLYCOL 90—Propylene glycol monolaurate
MAGNASWEET—monoammonium glyrrhizinate
PECEOL—Glyceryl monooleate
PLASDONE—polyvinylpyrrolidone
PLURONIC F68—Polyoxyethylene-polyoxypropylene block copolymer
PLURONIC F88—Polyoxyethylene glycol-polypropylene glycol block copolymer
PLURONIC F108—Poly(ethylene glycol)-block-poly(propylene glycol)-block-poly(ethylene glycol), PEG-PPG-PEG
POLOXAMINE 908—Oxirane, 2-methyl-, polymer with oxirane, ether with (1,2-ethanediyldinitrilo)tetrakis(propanol) (4:1)
PRECIROL—Glyceryl Palmitostearate
PROSWEET—syrup made of fructose, glucose, and sucrose, similar to honey
TRANSCUTOL—purified diethylene glycol monoethyl ether (DEGEE))
TWEEN 60—CAS9005-67-8, Polyoxyethylene sorbitan monostearate
TWEEN 80—CAS9005-65-6, Polysorbate 80

### Chemical Description

The structure of epinephrine (adrenaline) is

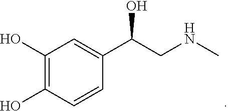

The IUPAC name for epinephrine is 4-[(1R)-1-hydroxy-2-(methylamino)ethyl]benzene-1,2-diol, CAS Reg. No. 329-65-7. Epinephrine has many prodrugs including those disclosed by Hussain and Truelove in U.S. Pat. No. 3,809,714, which is incorporated by reference for its disclosure of epinephrine prodrugs, their pharmaceutically acceptable salts, and methods of preparation of epinephrine prodrugs.

The structure of dipivefrin is

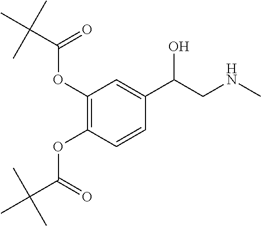 [SMILES: CNCC(O)c1ccc(OC(=O)C(C)(C)C)c(OC(=O)C(C)(C)C)c1]

The IUPAC name of dipivefrin is 4-(1-hydroxy-2-(methylamino)ethyl)-1,2-phenylene bis(2,2-dimethylpropanoate). The synonyms of dipivefrin are [±]-3,4-Dihydroxy-α-[(methylamino)methyl]benzyl alcohol 3,4-dipivalate, 1-(3′,4′-Dipivaloyloxyphenyl)-2-methylamino-1-ethanol, 4-[1-Hydroxy-2-(methylamino)ethyl]-O-phenylene dipivalate, Dipivalyl Epinephrine, propine, pivalephrine, and [2-(2,2-Dimethylpropanoyloxy)-4-[1-hydroxy-2-(methylamino)ethyl]phenyl]2,2-dimethylpropanoate. Dipivefrin has CAS Reg. No. 52365-63-6. Dipivefrin hydrochloride has CAS Reg. No. 64019-93-8. L-dipivefrin hydrochloride has the structure

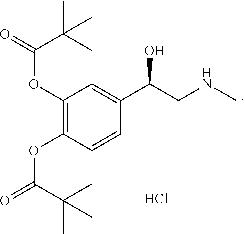 [SMILES: CNC[C@H](O)c1ccc(OC(=O)C(C)(C)C)c(OC(=O)C(C)(C)C)c1 ; Cl]

The epinephrine or epinephrine prodrug, e.g. dipivefrin, used in any of the methods disclosed herein can be the freebase or a pharmaceutically acceptable salt thereof. Preferably epinephrine or an epinephrine prodrug salt is an acid addition salt, for example, dipivefrin hydrochloride. The dipivefrin can be racemic dipivefrin or optically purified D- or L-dipivefrin, preferably L-dipivefrin.

Furthermore, the epinephrine or epinephrine prodrug, e.g. dipivefrin can be isotopically labeled with a pharmaceutically acceptable isotopic label. Examples of isotopes suitable for inclusion in the isotopically labeled dipivefrin, or salt or derivative thereof, include isotopes of hydrogen, such as ²H and ³H; carbon, such as ¹¹C, ¹³C and ¹⁴C; chlorine, such as ³⁶Cl; nitrogen, such as ¹³N and ¹⁵N; and oxygen, such as ¹⁵O, ¹⁷O and ¹⁸O. Isotopically labeled dipivefrin can be prepared by conventional techniques known to those skilled in the art.

Certain isotopically labeled epinephrine or epinephrine prodrug forms, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, 150 and ¹³N, can be useful in Positron Emission Topography (PET) studies for examining substrate receptor occupancy.

The epinephrine prodrug can also be a compound of the formula (I)

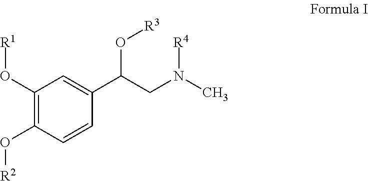

or a pharmaceutically acceptable salt thereof wherein
R¹, R², R³, and R⁴ are independently chosen from H, C₁-C₁₆alkylcarbonyl, mono or di-C₁-C₁₆alkylaminocarbonyl, C₁-C₁₆alkoxycarbonyl, (C₃-C₆cycloalkyl)C₀-C₄alkylcarbonyl, phenylC₀-C₄alkylcarbonyl, (C₀-C₁₆alkyl)sulfate, and (C₀-C₁₆alkyl)phosphate (naming into the point of attachment);
where each alkyl or alkoxy in the definition of R³ optionally contains one or more double or triple bonds, has one or more CH₂ groups replaced by O, S, NH, or NR⁵ where R⁵ is C₁-C₆alkyl, and is optionally substituted with one or more substituents independently chosen from halogen, hydroxyl, nitro, amino, oxo, or cyano;
where the R¹ alkyl or alkoxy may be joined to the R² alkyl or alkoxy to form a 5 to 7-membered ring or the R³ alkyl or alkoxy may be joined to the R⁴ alkyl or alkoxy to form a 5- to 7-membered ring; and
where not all of R¹, R², R³, and R⁴ are H, and dipivefrin (R¹ and R²=pivaloyl group, R³=R⁴=H) is excluded.

In preferred embodiments, R³ and R⁴ are H and each R¹ and R², independently, can be C₁-C₁₆acyl, for example, ethanoyl, n-propanoyl, isopropanoyl, n-butanoyl, isobutanoyl (also known as 2-methylpropanoyl or isobutyroyl or isobutyryl), sec-butanoyl, tert-butanoyl, n-pentanoyl, isopentanoyl, sec-pentanoyl, tert-pentanoyl, or neopentanoyl.

In certain circumstances, each R¹ and R² is the same and is not H and R³ and R⁴ are H. In certain circumstances, both R¹ and R² are not pivaloyl.

In a preferred embodiment, both R¹ and R² are isobutyryl and R³ and R⁴ are H. and the epinephrine prodrug of Formula I is L-diisobutyryl epinephrine, also known as dibutepinephrine.

The pharmaceutical agent in the formulation of this disclosure, the epinephrine or epinephrine prodrug, e.g. dipivefrin can be a pharmaceutically acceptable salt, solvate, clathrate, or polymorph, e.g. dipivefrin hydrochloride. The pharmaceutical agent, if chiral, can be racemic or a resolved isomer, e.g. L-dipivefrin hydrochloride.

Administration of dipivefrin has been found effective for the safe and rapid systemic delivery of epinephrine to a subject. Administration of dipivefrin can be by any appropriate route, for example oral administration or injection or intranasal or inhalation. Various diseases are amenable to treatment with systemic epinephrine, such as Type I allergic reactions including anaphylaxis and uticaria, or the disease can be cancer or microbial infection. However safe and convenient means of dosing epinephrine to individuals has been problematic. Epinephrine administration has been limited to administration by injection and nasal inhalation for lack of oral absorption, routes that are less convenient and less desirable than oral dosing. Furthermore, injectable epinephrine dosage forms are not heat and cold stable and must be maintained at the very narrow temperature range of 20° C. to 25° C. to prevent degradation at warmer temperatures and or product failures at cold temperature.

The inventor has surprisingly discovered a liquid non-aqueous dipivefrin formulation that can safely and effectively deliver epinephrine to the systemic circulation when taken orally. Oral transmucosal administration that avoids the GI tract provides mean plasma levels of epinephrine comparable to those provided by injectable epinephrine within 30, 15, or 5 minutes of administration. While previous dipivefrin orally disintegrating tablet formulations provided epinephrine plasma similar to those provided by injectable epinephrine in dogs, absorption of dipivefrin from these formulations were not as effective in humans despite humans having similar circulating levels of the esterase enzymes required to convert the prodrug dipivefrin to epinephrine.

Surprisingly, oral administration of dipivefrin, for example as an oral spray, oral film, liquid filled fast dissolve capsule, or buccal, lingual, or sublingual drop formulation, has been found effective for the safe and rapid systemic delivery of epinephrine to a subject and for treatment of type I allergy including anaphylaxis and urticaria, cancer, and microbial infection. This disclosure provides non-aqueous liquid epinephrine and epinephrine prodrug formulations that provide therapeutically effective amounts of epinephrine with a Tₘₐₓ similar to that of injectable epinephrine that are exceptionally heat/cold stable.

### Stable Formulations

The disclosure provides heat and cold stable epinephrine and epinephrine prodrug formulations. The formulations of this disclosure are stable at temperatures from −20° C. to 70° C., from −10° C. to 70° C., from 0° C. to 70° C., from 5° C. to 70° C., from 10° C. to 70° C., −20° C. to 65° C., from −10° C. to 65° C., from 0° C. to 65° C., 5° C. to 65° C., 10° C. to 65° C., −20° C. to 60° C., from −10° C. to 60° C., from 0° C. to 60° C., from 5° C. to 60° C., from 10° C. to 60° C., from −20° C. to 50° C., from −10° C. to 50° C., or from 0° C. to 50° C., from 5° C. to 50° C., from 10° C. to 50° C., from −20° C. to 45° C., from −10° C. to 45° C., or from 0° C. to 45° C., from 5° C. to 45° C., from 10° C. to 45° C., from −20° C. to 40° C., from −10° C. to 40° C., or from 0° C. to 40° C., from 5° C. to 40° C., from 10° C. to 40° C. for at least 10 days, at least 20 days, at least 30 days, at least 40 days, at least 50 days, at least 60 days, at least 65 days, at least 70 days, at least 85 days, at least 90 days, at least 4 months, at least 5 months, or at least 6 months, at least 9 months, at least 1 year, at least 18 months, or at least 2 years. Stability is characterized by lack of substantial change in the pharmaceutical agent and is characterized by absence of substantial increase in impurities formed from the degradation of the pharmaceutical agent. The increase of any one degradation impurity in a stable formulation is not more than (NMT) 2%, NMT 1.5%, NMT 1% and preferably NMT 0.5% from the initial values. The total increase of degradation impurities in a stable formulation is NMT than 10% and preferably NMT 5.0%, or 3.0%, or 2.0%, or 1.0% from the initial amount. The amounts of impurities can be measured by any suitable analytical method such as mass spectrometry or HPLC. In addition to lack of substantial degradation, stability is also characterized by lack of substantial change in physical state of the formulation, for example, change from a liquid to a solid when temperature drops below a certain point, such as below 0° C. In certain circumstances, stability is characterized by potency not dropping by more than 20% from initial value and preferably not more than 10% from initial value. The potency can be measured by any suitable analytical method such as HPLC.

The potential degradation impurities for dipivefrin are shown in the “Table of Potential Degradants in Dipivefrin Formulations,” which follows. A heat stable dipivefrin formulation is one in which an increase of NMT 1.0% and preferable NMT 0.5% of any of the degradants listed in the table are detected after storage at 60° C. for at least 10 days, at least 20 days, at least 30 days, at least 40 days, at least 50 days, at least 60 days, at least 65 days, at least 70 days, at least 85 days, at least 90 days, at least 3 months. Cold stable epinephrine and epinephrine prodrug formulations are the ones that do not freeze at 0° C., or at −5° C., or at −10° C. or at −15° C. or at −20° C.

Table of Potential Degradants in Dipivefrin Formulations

| Com- | | |
|---|---|---|
| pound | Structure | Name |
| IN- 003 | 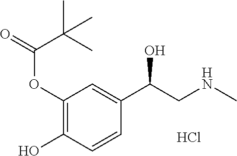 [SMILES: CNC[C@H](O)c1ccc(O)c(OC(=O)C(C)(C)C)c1 ; Cl] | 3- monopivaloyl- epinephrine |
| IN- 004 | 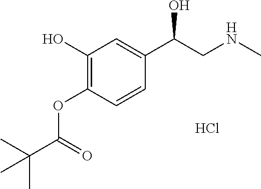 [SMILES: CNC[C@H](O)c1ccc(OC(=O)C(C)(C)C)c(O)c1 ; Cl] | 4- monopivaloyl- epinephrine |
| IN- 006 | 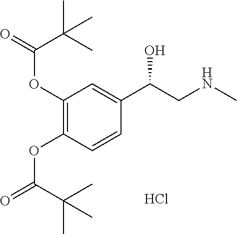 [SMILES: CNC[C@@H](O)c1ccc(OC(=O)C(C)(C)C)c(OC(=O)C(C)(C)C)c1 ; Cl] | D-Dipivefrin |
| IN- 010 | 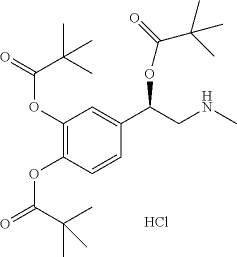 [SMILES: CNC[C@H](OC(=O)C(C)(C)C)c1ccc(OC(=O)C(C)(C)C)c(OC(=O)C(C)(C)C)c1 ; Cl] | L- dipivefrin pivalate |
| IN- 011 | 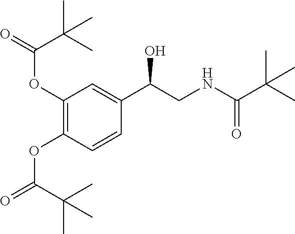 [SMILES: CC(C)(C)C(=O)NC[C@H](O)c1ccc(OC(=O)C(C)(C)C)c(OC(=O)C(C)(C)C)c1] | Pivaloyl amide |

The disclosure also includes epinephrine and epinephrine prodrug formulations that are stable at ambient temperatures and ambient humidity for at least 1 month, 2 months, 3 months, 4 months, 5 months, 6 months, 9 months, 12 months, 18 months, 2 years, or up to at least 3 years. The same impurity criteria apply for heat/cold stability.

### Methods of Treatment

The present disclosure provides compositions and methods related to the use of non-aqueous epinephrine and epinephrine prodrug formulations, e.g. dipivefrin hydrochloride formulations, for treating a breathing difficulty, type I allergic reactions (also called type I hypersensitivity reaction) including anaphylaxis and urticaria, cancer, or a microbial infection in a subject, preferably a human subject, in need of such treatment. In addition, the present disclosure provides novel therapeutic approaches to treating type I allergic reactions including anaphylaxis and urticaria or to treating cancer or a microbial infection based upon therapeutic regimens utilizing epinephrine or an epinephrine prodrug alone, as a monotherapy, or in combination with at least one additional therapeutic agent, such as an anticancer agent or an antimicrobial agent.

A “breathing difficulty” includes any disease or disorder that causes a sensation of uncomfortable or abnormal breathing, e.g., shortness of breath. For the purposes of this disclosure physical obstruction of the airway are not included in the definition of “a breathing difficulty.” Breathing difficulties include difficulty breathing or shortness of breath due to anaphylaxis, asthma, bronchitis, emphysema, croup, COPD, or a respiratory infection.

Type I allergic reactions, or type I hypersensitivity reactions can include any of the following conditions allergic asthma, allergic conjunctivitis, allergic rhinitis, anaphylaxis, angioedema, urticaria, eosinophilia, drug allergy and food allergy.

In certain embodiments, the type-1 hypersensitivity reaction (systemic allergic reaction) is anaphylaxis.

Symptoms of anaphylaxis include hives, generalized itching, nasal congestion, wheezing, difficulty breathing, cough, cyanosis, lightheadedness, dizziness, confusion, slurred speech, rapid pulse, palpitations, nausea and vomiting, abdominal pain or cramping, skin redness or inflammation, nasal flaring, and intercostal retractions.

Symptoms of type-1 allergic reaction can include any of the following generalized hives (urticaria), itching (pruritus), flushing, swelling (angioedema) of the afflicted tissues, a burning sensation of the skin (common in those with angioedema), swelling of the tongue or throat, respiratory symptoms such as shortness of breath, wheezes, or stridor shortness of breath, coronary artery spasm, myocardial infarction, dysrhythmia, or cardiac arrest (those with underlying coronary disease are at greater risk of cardiac effects), tachycardia, bradycardia, and a Bezold-Jarisch reflex.

In one aspect, the present disclosure provides a method for treating anaphylaxis, type I allergic reactions including anaphylaxis and urticaria, and for treating cancer or a microbial infection, the method comprising orally administering to a subject in need thereof of non-aqueous epinephrine or epinephrine prodrug formulations, e.g. a non-aqueous dipivefrin hydrochloride formulation comprising dipivefrin, or a pharmaceutically acceptable salt, solvate, clathrate, hydrate, polymorph, analog or derivative thereof. In one embodiment, the dipivefrin formulation comprises dipivefrin freebase or dipivefrin hydrochloride

The disclosure provides a method of for treating a type-I allergic reaction comprising administering an epinephrine or epinephrine prodrug formulation of the disclosure to a patient experiencing such an allergic reaction wherein the formulation provides a plasma epinephrine concentration in the patient sufficient for the alleviation of one, more than one or all of the symptoms of the type I allergic reaction in the patient. The disclosure provides a method of for treating a type-I allergic reaction comprising administering an epinephrine or epinephrine prodrug formulation of the disclosure to a patient experiencing such an allergic reaction wherein the formulation provides a plasma epinephrine concentration in the patient sufficient to prevent the progression of more than one or all of the symptoms of the type I allergic reaction in the patient. The formulation can be a formulation comprising an epinephrine prodrug, where the epinephrine prodrug is L-dipivefrin hydrochloride.

Disclosed herein is a method for systemic delivery of epinephrine to a subject, comprising orally administering a non-aqueous epinephrine or epinephrine prodrug formulation to a subject.

Also disclosed herein is a method of treatment of a disease amenable to treatment by in vivo delivery of systemic epinephrine to a subject in need thereof. The method comprises administering a non-aqueous epinephrine prodrug formulation to a subject in need of in vivo delivery of systemic epinephrine. Non-limiting examples of such diseases include allergic reactions, anaphylaxis, cancer, and microbial infections. Administration of the epinephrine prodrug can be performed by any suitable oral route, including as an oral spray, an oral film, a liquid-filled capsule, or a buccal, lingual, or sublingual liquid, e.g., a droplet. The oral spray and oral film can be placed on the tongue, under the tongue (sublingual) or in the buccal space. Preferably, the oral spray is a sublingual spray. The amount of pharmaceutical agent administered depends on the mammal being treated. For human patients the unit dose or daily dosage will normally be determined by the prescribing physician with the dosage generally varying according to the age, sex, diet, weight, general health and response of the individual patient, the severity of the patient's symptoms, the precise indication or condition being treated, the severity of the indication or condition being treated, time of administration, route of administration, the disposition of the composition, rate of excretion, drug combination, and the discretion of the prescribing physician. Also, the route of administration may vary depending on the condition and its severity. Preferably, the pharmaceutical composition is in unit dosage form. In such form, the preparation is subdivided into unit doses containing appropriate quantities of the pharmaceutical agent, e.g., an effective amount to achieve the desired purpose. The amount and frequency of administration of the compounds described herein, and if applicable other therapeutic agents and/or therapies will be regulated according to the judgment of the attending clinician (physician) considering such factors as described above. Thus, the amount of pharmaceutical composition to be administered may vary widely. Administration may occur in an amount of between about 0.001 mg/kg of body weight to about 1000 mg/kg of body weight per day (administered in single or divided doses), or e.g., at least about 0.1 mg/kg of body weight per unit dose.

The disclosure includes a method for treating type I allergic reactions including anaphylaxis and uticaria. The method can comprise administering non-aqueous oral epinephrine or epinephrine prodrug formulation of the disclosure to a subject experiencing type I allergic reactions including anaphylaxis and uticaria. The method also comprises administering a therapeutically effective amount of non-aqueous epinephrine or epinephrine prodrug formulation of the disclosure to a subject experiencing a less severe allergic reaction. In preferred embodiments non-aqueous epinephrine or epinephrine prodrug formulation of the disclosure is administered orally, for example in an oral spray, oral film, liquid-filled capsule, or a sublingual, lingual, or buccal drop. The oral spray and oral film can be placed on the tongue, under the tongue (sublingual) or in the buccal space. Preferably, the oral spray is a sublingual spray. In one embodiment, the formulation comprises dipivefrin freebase or dipivefrin hydrochloride. The non-aqueous oral epinephrine or epinephrine prodrug formulation can be administered in an amount sufficient to provide a therapeutically effective plasma level of epinephrine in the subject. For example an amount of dipivefrin sufficient to provide an epinephrine plasma Cₘₐₓ of 0.05 to 50 ng/mL, 0.1 to 50 ng/mL, 0.05 to 25 ng/mL 0.1 to 25 ng/mL, 0.05 to 10 ng/mL, 0.1 to 10 ng/mL, 0.05 to 5 ng/mL, 0.1 to 5 ng/mL, 0.05 to 1 ng/mL, 0.1 to 1 ng/mL. For treatment of anaphylaxis an epinephrine plasma concentration of at least 0.05 ng/mL, 0.1 ng/mL, 0.2 ng/mL, 0.25 ng/ml, 0.3 ng/mL, 0.4 ng/mL, or 0.5 ng/mL should be achieved in less than 30 min, and preferably in less than 15 minutes, or 10 minutes or 5 minutes postdose. The disclosure provides epinephrine plasma concentration of at least 0.05 ng/mL, 0.1 ng/mL, 0.2 ng/mL, 0.25 ng/mL 0.3 ng/mL, 0.35 ng/mL or 0.4 ng/mL in the first 15 minutes after dosing.

In one embodiment, the non-aqueous oral epinephrine or epinephrine prodrug formulations that provides the desired Cₘₐₓ with a unit dosage of less than 20 mg epinephrine or epinephrine prodrug, less than 10 mg epinephrine or epinephrine prodrug, and in some embodiments with a unit dosage of less than 5 mg epinephrine or epinephrine prodrug.

The method can comprise administering to the subject non-aqueous oral epinephrine or epinephrine prodrug formulation of the disclosure less than 1 min, less than 2 min., less than 5 min., less than 10 min., less than 20 min, or less than 30 min after the onset of anaphylaxis.

The method can comprise administering to the subject a therapeutically effective amount of non-aqueous oral epinephrine or epinephrine prodrug formulation of the disclosure 6 hours before, 5 hours before, 4 hours before, 3 hours before, 2 hours before, 1 hour before, 30 min before, 10 min before, or 5 min before a potential exposure to an anaphylaxis trigger for the subject.

An “anaphylaxis trigger” means a substance that will likely cause anaphylactic reaction in the subject. Examples of anaphylaxis triggers include food to which a pe, such as peanuts, tree nuts, fish, milk; certain medications, such as antibiotics (penicillins and cephalosporins) and analgesics (aspirin, ibuprofen); venom from insects, including bees, yellow jackets, wasps, hornets, and fire ants; and latex from natural rubber.

Also provided are formulations and devices for use in treating conditions mediated by adrenergic receptors, and/or one or more symptoms thereof, and methods of treatment of such conditions comprising administering the formulations and using the devices disclosed herein.

In certain embodiments, the condition is (1) treatment of acute hypersensitivity, such as a type-1 hypersensitivity reaction (for example, such as an anaphylactoid reaction (systemic allergic reaction) to foods, drugs, animal serums, insect bites and stings, and other allergens (see below)), (2) treatment of acute asthmatic attacks to relieve bronchospasm not controlled by inhalation or subcutaneous administration of other solutions of the drug, (3) treatment and prophylaxis of cardiac arrest and/or attacks of transitory atrioventricular (A-V) heart block with syncopal seizures (Stokes-Adams Syndrome, (4) to increase mean arterial blood pressure in adult patients with hypotension associated with septic shock, (5) for induction and maintenance of mydriasis during intraocular surgery.

In certain embodiments, the type-1 hypersensitivity reaction (systemic allergic reaction) is chosen from allergic asthma, allergic conjunctivitis, allergic rhinitis (hay fever), anaphylaxis, angioedema, urticaria (hives), eosinophilia, antibiotic allergy (e. g., to penicillin or cephalosporin), and food allergy (e. g., to peanuts or shellfish). In certain embodiments, the type-1 hypersensitivity reaction (systemic allergic reaction) is anaphylaxis. Symptoms of anaphylaxis include hives, generalized itching, nasal congestion, wheezing, difficulty breathing, cough, cyanosis, lightheadedness, dizziness, confusion, slurred speech, rapid pulse, palpitations, nausea and vomiting, abdominal pain or cramping, skin redness or inflammation, nasal flaring, and intercostal retractions.

In certain embodiments, the symptom of the type-1 hypersensitivity reaction (systemic allergic reaction) is chosen from generalized hives (urticaria), itching (pruritus), flushing, swelling (angioedema) of the afflicted tissues, a burning sensation of the skin (common in those with angioedema), swelling of the tongue or throat, respiratory symptoms such as shortness of breath, wheezes, or stridor shortness of breath, coronary artery spasm, myocardial infarction, dysrhythmia, or cardiac arrest (those with under lying coronary disease are at greater risk of cardiac effects), tachycardia, bradycardia, and a Bezold-Jarisch reflex.

In certain embodiments, the type-1 hypersensitivity reaction (systemic allergic reaction) is caused by stinging insects (e. g., order Hymenoptera, which include bees, wasps, hornets, yellow jackets and fire ants), biting insects (e. g., triatoma, mosquitoes), allergen immunotherapy, foods, drugs, diagnostic testing substances (e. g., radiocontrast media) and other allergens, as well as idiopathic anaphylaxis or exercise-induced anaphylaxis.

In certain embodiments, cardiac arrest is out of hospital cardiac arrest. Also provided are embodiments wherein any embodiment above may be combined with any one or more of these embodiments, provided the combination is not mutually exclusive.

### Methods for Treating Cancer

The disclosure includes a method for treating cancer comprising administering an non-aqueous oral epinephrine or epinephrine prodrug formulation of the disclosure to a subject in need of treatment of a cancer. The cancer can be, for example, a brain cancer, a glioma, a sarcoma, a skin cancer, a breast cancer, a lung cancer, a non-small-cell lung cancer, a mesothelioma, an appendicular cancer, a genitourinary cancer, a renal cell carcinoma, a prostate cancer, a bladder cancer, a testicular cancer, a penile cancer, a cervical cancer, an ovarian cancer, a von Hippel Lindau disease, a head and neck cancer, a gastrointestinal cancer, a hepatocellular carcinoma, a gallbladder cancer, an esophageal cancer, a gastric cancer, a colorectal cancer, a pancreatic cancer, a neuroendocrine tumor, a thyroid tumor, a pituitary tumor, an adrenal tumor, a hematological malignancy, a lymphoma, a leukemia, or a combination thereof. Skin cancer can be a melanoma, a basal cell cancer, or a squamous cell skin carcinoma.

In one embodiment, the cancer is renal cell carcinoma.

In one embodiment, the cancer is breast cancer.

In one embodiment, the cancer is acute myeloid leukemia (AML) or acute lymphoblastic leukemia (ALL).

In one embodiment, cancer is a B-cell lymphoma.

In one embodiment, the cancer is a non-Hodgkins B-cell lymphoma.

In one embodiment, cancer is a glioma.

The method for treating cancer can be combination therapy. As used herein, “combination therapy” includes administration of an oral non-aqueous oral epinephrine or epinephrine prodrug formulation of the disclosure in which epinephrine or an epinephrine prodrug, e.g. dipivefrin hydrochloride, is a first pharmaceutical agent, together with at least one additional pharmaceutical agent, e.g. an anti-cancer agent. In a combination method of this disclosure, the epinephrine, which is either directly administered or generated in vivo from the metabolism of a prodrug, is used as part of a specific treatment regimen intended to provide a beneficial effect from the co-action of the epinephrine and the additional anticancer treatment.

The additional anticancer treatment can be surgery, chemotherapy, radiation, endocrine therapy, transplantation of stem cells, a molecularly-targeted therapy, or a biological therapy. Examples of chemotherapeutics include anthracyclines such as doxorubicin and daunoribicin, taxanes such as paclitaxel and docetaxel, and platinum-based chemotherapies such as cisplatin and oxaliplatin. Examples of an endocrine therapy include tamoxifen, aromatase inhibitors, and androgen deprivation therapy for prostate cancer. Examples of a molecularly targeted therapy include hormone therapies, signal transduction inhibitors, gene expression modulators, apoptosis inducers, angiogenesis inhibitors, immunotherapies, monoclonal antibodies that deliver toxic molecules, cancer vaccines, and gene therapy. Examples of biological therapy include monoclonal antibodies, or MAbs, cytokines, cancer treatment vaccines, Bacillus Calmette-Guérin therapy (BCG), oncolytic virus therapy, gene therapy, and adoptive T-cell transfer therapy.

In one embodiment, the at least one additional anticancer treatment can be an immunotherapy that uses certain parts of a person's immune system to fight diseases such as cancer. Examples of an immunotherapy include monoclonal antibodies, immune checkpoint inhibitors, cancer vaccines, cytokines and immunomodulating drugs (or IMiDs), Bacille Calmette-Guérin (BCG), imiquimod, and combinations thereof. Examples of the adoptive cell transfer (ACT) therapy include a CAR (chimeric antigen receptor) modified T-cell therapy such as tisagenlecleucel and CAR modified NK cell therapy. In one embodiment, the cancer vaccine is sipuleucel-T, approved for prostate cancer in the United States.

In one embodiment, a combination therapy of the disclosure comprises administering at least one additional pharmaceutical agent to the subject. The at least one additional pharmaceutical agent may be administered in a single dosage form with the oral non-aqueous oral epinephrine or epinephrine prodrug formulation, or in a separate dosage form from the oral non-aqueous oral epinephrine or epinephrine prodrug formulation. In one embodiment, the at least one additional pharmaceutical agent is selected from the group consisting of an alkylating agent, an intercalating agent a tubulin binding agent, a corticosteroid, and combinations thereof.

The at least one additional pharmaceutical agent may be a therapeutic agent, for example an anti-cancer agent or a cancer chemotherapeutic agent, a non-therapeutic agent, or combinations thereof. With respect to therapeutic agents, the beneficial effect of the combination includes, but is not limited to, pharmacokinetic or pharmacodynamics co-action resulting from the combination of therapeutically active compounds. With respect to non-therapeutic agents, the beneficial effect of the combination may relate to the mitigation of toxicity, a side effect, or an adverse event associated with a therapeutically active agent in the combination.

In one embodiment, the at least one additional pharmaceutical agent is a therapeutic agent. In one embodiment, the therapeutic agent is an anti-cancer agent. In one embodiment, the anticancer agent is a Bruton's tyrosine kinase (BTK) inhibitor such as ibrutinib. In one embodiment, an oral non-aqueous oral epinephrine or epinephrine prodrug formulation of the disclosure is administered along with ibrutinib in a single dosage form or in separate dosage forms. In one embodiment, the dosage form of the additional pharmaceutical agent is an oral dosage form. In another embodiment, the dosage form of the additional pharmaceutical agent is suitable for intravenous administration.

In one embodiment, the anti-cancer agent is a drug that is approved for use in treating lymphoma. Non-limiting examples of such drugs include belinostat, bendamustine hydrochloride, bleomycin, bortezomib, brentuximab vedotin, carmustine, chlorambucil, cyclophosphamide, denileukin diftitox, liposomal cytarabine, doxorubicin hydrochloride, ibritumomab tiuxetan, ibrutinib, idelalisib, lenalidomide, liposomal cytarabine, mechlorethamine hydrochloride, methotrexate, nelarabine, perixafor, pralatrexate, prednisone, recombinant interferon Alfa-2b, rituximab, romidepsin, tositumomab, iodine I-131 tositumomab, vinblastine sulfate, vincristine sulfate, and vorinostat.

In one embodiment, the method is a method of treating a lymphoma using a combination therapy comprising a of the oral non-aqueous epinephrine or epinephrine prodrug formulation of this disclosure and a chemotherapy regimen for the treatment of the lymphoma. In one embodiment, the chemotherapy regimen is the CHOP regimen (cyclophosphamide, doxorubicin (hydroxydaunorubicin), vincristine (Oncovin), and prednisone). In another embodiment, the chemotherapy regimen is selected from COPP, CVP, EPOCH, Hyper-CVAD, ICE, R-CHOP, and R-CVP.

In one embodiment, the anti-cancer agent is selected from an inhibitor of EZH2, e.g., EPZ-6438. In one embodiment, the anti-cancer agent is selected from taxol, vincristine, doxorubicin, temsirolimus, carboplatin, ofatumumab, rituximab, and combinations thereof.

In one embodiment, the at least one additional pharmaceutical agent is a B cell receptor pathway inhibitor. In some embodiments, the B cell receptor pathway inhibitor is a CD79A inhibitor, a CD79B inhibitor, a CD 19 inhibitor, a Lyn inhibitor, a Syk inhibitor, a P13K inhibitor, a Blnk inhibitor, a PLCy inhibitor, a PKCP inhibitor, or a combination thereof. In some embodiments, the at least one additional pharmaceutical agent is an antibody, B cell receptor signaling inhibitor, a PI3K inhibitor, an IAP inhibitor, an mTOR inhibitor, a radioimmunotherapeutic, a DNA damaging agent, a proteasome inhibitor, a histone deacetylase inhibitor, a protein kinase inhibitor, a hedgehog inhibitor, an Hsp90 inhibitor, a telomerase inhibitor, a Jak1/2 inhibitor, a protease inhibitor, a PKC inhibitor, a PARP inhibitor, or a combination thereof.

In one embodiment, the at least one additional pharmaceutical agent is an inhibitor of the checkpoint signaling pathway involving the programmed death 1 (PD-1) receptor and its ligands (PD-L1/2). In one embodiment, the method comprises a combination of an anti-PD-L1 agent and an anti-PD-1 agent. In one embodiment, the inhibitor is an anti-PD-L1 agent selected from MPDL3280A (an engineered human monoclonal antibody targeting PD-L 1), MSB0010718C and MEDI473. In one embodiment, the inhibitor is an anti-PD-1 agent selected from CT-011/pidilizumab, nivolumab, pembrolizumab. In one embodiment, the inhibitor is selected from MPDL3280A, MSB0010718C, MED1473, CT-011/pidilizumab nivolumab, and pembrolizumab, and combinations of any two or more of the foregoing. In one embodiment, the inhibitor is selected from ipilimumab, pembrolizumab, nivolumab, atezolizumab, avelumab, durvalumab, and combinations thereof. In one embodiment, the inhibitor is an anti-CTLA-4 antibody. An example of anti-CTLA-4 antibody is Ipilimumab (trade name Yervoy).

In one embodiment, the at least one additional pharmaceutical agent is a therapeutic agent selected from the group consisting immunomodulatory drugs (IMids) capable of stimulating both NK cells and T-cells such as thalidomide, lenalidomide, and pomalidomide. In one embodiment, the combination therapy also includes an anti-inhibitory KIR antibody (IPH-2102).

In one embodiment, the at least one additional pharmaceutical agent is a therapeutic agent selected from inhibitors of indoleamine-2,3-dioxygenase (IDO). In one embodiment, the inhibitor is selected from the group consisting of indoximod, epacadostat, NLG 919, IDO1-derived peptide, epacadostat, GDC0919 or a combination thereof.

In one embodiment, the at least one additional pharmaceutical agent is a therapeutic agent selected from the group consisting of ibrutinib, rituximab, doxorubicin, prednisolone, vincristine, velcade, and everolimus, and combinations thereof. In one embodiment, the at least one additional pharmaceutical agent is a therapeutic agent selected from cyclophosphamide, hydroxydaunorubicin (also referred to as doxorubicin or Adriamycin™) vincristine (also referred to as Oncovin™), prednisone, prednisolone, and combinations thereof. In one embodiment, the at least one additional pharmaceutical agent is a therapeutic agent selected from the group consisting of nivolumab, atezolizumab, avelumab, durvalumab, CT-011/pidilizumab, and pembrolizumab, and combinations of two or more of any of the foregoing.

In one embodiment, the at least one additional pharmaceutical agent is selected from chlorambucil, ifosphamide, doxorubicin, mesalazine, thalidomide, lenlidomide, temsirolimus, everolimus, fludarabine, fostamatinib, paclitaxel, docetaxel, ofattumumab, rituximab, dexamethasone, prednisone, CAL-101, ibritumomab, tositumomab, bortezomib, pentostatin, endostatin, or a combination thereof.

In one embodiment, the at least one additional pharmaceutical agent is a monoclonal antibody such as, for example, alemtuzumab, bevacizumab, catumaxomab, cetuximab, edrecolomab, gemtuzumab, ofatumumab, panitumumab, rituximab, trastuzumab, eculizumab, efalizumab, muromab-CB3, natalizumab, adalimumab, afelimomab, certolizumab pegol, golimumab, infliximab, basiliximab, canakinumab, daclizumab, mepolizumab, tocilizumab, ustekinumab, ibritumomab tiuxetan, tositumoma, abagovomab, adecatumumab, alemtuzumab, anti-CD30 monoclonal antibody Xmab2513, anti-MET monoclonal antibody MetMab, apolizumab, apomab, arcitumomab, basiliximab, bispecific antibody 2B1, blinatumomab, brentuximab vedotin, capromab pendetide, cixutumumab, claudiximab, conatumumab, dacetuzumab, denosumab, eculizumab epratuzumab, ertumaxomab, etaracizumab, figitumumab, fresolimumab, galiximab, ganitumab, gemtuzumab ozogamicin, glembatumumab, ibritumomab, inotuzumab ozogamicin, ipilimumab, lexatumumab, lintuzumab, lintuzumab, lucatumumab, mapatumumab, matuzumab, milatuzumab, monoclonal antibody CC49, necitumumab, nimotuzumab, ofatumumab, oregovomab, pertuzumab, ramacurimab, ranibizumab, siplizumab, sonepcizumab, tanezumab, tositumomab, trastuzumab, tremelimumab, tucotuzumab celmoleukin, veltuzumab, visilizumab, volociximab, and zalutumumab, rituximab, certuximab, daraumumab, ublituximab, ocaratuzumab, obinutuzumab.

In one embodiment, the additional pharmaceutical agent is a standard chemotherapy regimen that comprises one or more therapeutic agents selected from the group consisting of ibrutinib, rituximab, doxorubicin, prednisolone, vincristine, velcade, cyclophosphoamide, dexamethasone and everolimus. In one embodiment, the standard chemotherapy regimen is selected from CHOP, (cyclophosphamide, hydroxydaunorubicin, and prednisone or prednisolone), COOP (cyclophosamide, vincristine sulfate, prednisone), EPOCH (etoposide, prednisone, vincristine sulfate, cyclophosphamide, doxorubicin hydrochloride, Hyper-CVAD (cyclophosphamide, vincristine sulfate, doxorubicin hydrochloride, dexamethasone), ICE (ifosfamide, carboplatin, etoposide), R-CHOP (rituximab, cyclophosamide, vincristine sulfate, procarbazine hydrochloride, prednisone, and R-CVP (rituximab, cyclophosamide, vincristine sulfate, prednisone).

In an embodiment, the at least one additional pharmaceutical agent is a cytokine selected from the group consisting of interferons (INFs) and interleukins (ILs). Examples of interferons include INF-alfa. Examples of interleukins include IL-2 (aldesleukin), IL-6, IL-12, IL-15, and IL-21.

In an embodiment, the at least one additional pharmaceutical agent is selected from therapies approved for bladder cancer including atezolizumab (Tecentriq), avelumab (Bavencio), enfortumab vedotin-ejfv (Padcev), erdafitinib (Balversa), nivolumab (Opdivo), nogapendekin alfa inbakicept-pmln (Anktiva), pembrolizumab (Keytruda).

In an embodiment, the at least one additional pharmaceutical agent is selected from therapies approved for brain cancer including belzutifan (Welireg), bevacizumab (Avastin), dabrafenib (Tafinlar), everolimus (Afinitor), tovorafenib (Ojemda), trametinib (Mekinist), and vorasidenib (Voranigo).

In an embodiment, the at least one additional pharmaceutical agent is selected from therapies approved for breast cancer including abemaciclib (Verzenio), ado-trastuzumab emtansine (Kadcyla), alpelisib (Piqray), anastrozole (Arimidex), capivasertib (Trugap), datopotamab deruxtecan-dlnk (Datroway), elacestrant dihydrochloride (Orserdu), everolimus (Afinitor), exemestane (Aromasin), fam-trastuzumab deruxtecan-nxki (Enhertu), fulvestrant (Faslodex), goserelin acetate (Zoladex), inavolisib (Itovebi), lapatinib ditosylate (Tykerb), letrozole (Femara), margetuximab-cmkb (Margenza), neratinib maleate (Nerlynx), olaparib (Lynparza), palbociclib (Ibrance), pembrolizumab (Keytruda), pertuzumab (Perjeta), pertuzumab, trastuzumab, and hyaluronidase-zzxf (Phesgo), ribociclib (Kisqali), ribociclib succinate and letrozole (Kisqali Femara Co-Pack), sacituzumab govitecan-hziy (Trodelvy), talazoparib tosylate (Talzenna), tamoxifen citrate (Soltamox), toremifene (Fareston), trastuzumab (Herceptin), tucatinib (Tukysa).

In an embodiment, the at least one additional pharmaceutical agent is selected from therapies approved for cervical cancer including bevacizumab (Avastin), pembrolizumab (Keytruda), and tisotumab vedotin-tftv (Tivdak).

In an embodiment, the at least one additional pharmaceutical agent is selected from therapies approved for colorectal cancer including adagrasib (Krazati), bevacizumab (Avastin), cetuximab (Erbitux), encorafenib (Braftovi), fruquintinib (Fruzagla), ipilimumab (Yervoy), nivolumab (Opdivo), panitumumab (Vectibix), pembrolizumab (Keytruda), ramucirumab (Cyramza), regorafenib (Stivarga), sotorasib (Lumakras), tucatinib (Tukysa), and ziv-aflibercept (Zaltrap).

In an embodiment, the at least one additional pharmaceutical agent is selected from therapies approved for dermatofibrosarcoma protuberans including imatinib mesylate (Gleevec).

In an embodiment, the at least one additional pharmaceutical agent is selected from therapies approved for endocrine and neuroendocrine tumors including avelumab (Bavencio), everolimus (Afinitor), iobenguane I 131 (Azedra), lanreotide acetate (Somatuline Depot), and lutetium Lu 177-dotatate (Lutathera).

In an embodiment, the at least one additional pharmaceutical agent is selected from therapies approved for endometrial cancer including dostarlimab-gxly (Jemperli), durvalumab (Imfinzi), lenvatinib mesylate (Lenvima), and pembrolizumab (Keytruda).

In an embodiment, the least one additional pharmaceutical agent is selected from therapies approved for esophageal cancer including fam-trastuzumab deruxtecan-nxki (Enhertu), ipilimumab (Yervoy), nivolumab (Opdivo), pembrolizumab (Keytruda), ramucirumab (Cyramza), tislelizumab-jsgr (Tevimbra), trastuzumab (Herceptin), and zolbetuximab-clzb (Vyloy).

In an embodiment, the least one additional pharmaceutical agent is selected from therapies approved for head and neck cancer including cetuximab (Erbitux), nivolumab (Opdivo), pembrolizumab (Keytruda), and toripalimab-tpzi (Loqtorzi).

In an embodiment, the least one additional pharmaceutical agent is selected from therapies approved for gastrointestinal stromal tumor including avapritinib (Ayvakit), imatinib mesylate (Gleevec), regorafenib (Stivarga), ripretinib (Qinlock), and sunitinib malate (Sutent).

In an embodiment, the least one additional pharmaceutical agent is selected from therapies approved for giant cell tumor including denosumab (Xgeva), and pexidartinib hydrochloride (Turalio).

In an embodiment, the least one additional pharmaceutical agent is selected from therapies approved for kidney cancer including avelumab (Bavencio), axitinib (Inlyta), belzutifan (Welireg), bevacizumab (Avastin), cabozantinib-s-malate (Cabometyx), everolimus (Afinitor), ipilimumab (Yervoy), lenvatinib mesylate (Lenvima), nivolumab (Opdivo), pazopanib hydrochloride (Votrient), pembrolizumab (Keytruda), sorafenib tosylate (Nexavar), sunitinib malate (Sutent), temsirolimus (Torisel), and tivozanib hydrochloride (Fotivda).

In an embodiment, the least one additional pharmaceutical agent is selected from therapies approved for leukemia including acalabrutinib (Calquence), alemtuzumab (Campath), asciminib hydrochloride (Scemblix), avapritinib (Ayvakit), blinatumomab (Blincyto), bosutinib (Bosulif), brexucabtagene autoleucel (Tecartus), dasatinib (Sprycel), duvelisib (Copiktra), enasidenib mesylate (Idhifa), gemtuzumab ozogamicin (Mylotarg), gilteritinib fumarate (Xospata), glasdegib maleate (Daurismo), ibrutinib (Imbruvica), idelalisib (Zydelig), imatinib mesylate (Gleevec), inotuzumab ozogamicin (Besponsa), ivosidenib (Tibsovo), lisocabtagene maraleucel (Breyanzi), midostaurin (Rydapt), nilotinib (Tasigna), obecabtagene autoleucel (Aucatzyl), obinutuzumab (Gazyva) ofatumumab (Arzerra), olutasidenib (Rezlidhia), pemigatinib (Pemazyre), pirtobrutinib (Jaypirca), ponatinib hydrochloride (Iclusig), quizartinib dihydrochloride (Vanflyta), revumenib citrate (Revuforj), rituximab (Rituxan), rituximab and hyaluronidase human (Rituxan Hycela), tagraxofusp-erzs (Elzonris), tisagenlecleucel (Kymriah), tretinoin (Vesanoid), venetoclax (Venclexta), and zanubrutinib (Brukinsa).

In an embodiment, the least one additional pharmaceutical agent is selected from therapies approved for liver and bile duct including atezolizumab (Tecentriq), atezolizumab and hyaluronidase-tqjs (Tecentriq Hybreza), bevacizumab (Avastin), cabozantinib-s-malate (Cabometyx), durvalumab (Imfinzi), futibatinib (Lytgobi), ipilimumab (Yervoy), ivosidenib (Tibsovo), lenvatinib mesylate (Lenvima), nivolumab (Opdivo), pembrolizumab (Keytruda), pemigatinib (Pemazyre), ramucirumab (Cyramza), regorafenib (Stivarga), sorafenib tosylate (Nexavar), tremelimumab-actl (Imjudo), and zanidatamab-hrii (Ziihera).

In an embodiment, the least one additional pharmaceutical agent is selected from therapies approved for lung cancer including adagrasib (Krazati), afatinib dimaleate (Gilotrif), alectinib (Alecensa), amivantamab-vmjw (Rybrevant), atezolizumab (Tecentriq), atezolizumab and hyaluronidase-tqjs (Tecentriq Hybreza), bevacizumab (Avastin), binimetinib (Mektovi), brigatinib (Alunbrig), capmatinib hydrochloride (Tabrecta), cemiplimab-rwlc (Libtayo), ceritinib (Zykadia), crizotinib (Xalkori), dabrafenib mesylate (Tafinlar), dacomitinib (Vizimpro), durvalumab (Imfinzi), encorafenib (Braftovi), ensartinib hydrochloride (Ensacove), entrectinib (Rozlytrek), erlotinib hydrochloride (Tarceva), fam-trastuzumab deruxtecan-nxki (Enhertu), gefitinib (Iressa), ipilimumab (Yervoy), lazertinib mesylate hydrate (Lazcluze), lorlatinib (Lorbrena), necitumumab (Portrazza), nivolumab (Opdivo), osimertinib mesylate (Tagrisso), pembrolizumab (Keytruda), pralsetinib (Gavreto), ramucirumab (Cyramza), repotrectinib (Augtyro), selpercatinib (Retevmo), sotorasib (Lumakras), tarlatamab-dlle (Imdelltra), tepotinib hydrochloride (Tepmetko), trametinib dimethyl sulfoxide (Mekinist), tremelimumab-actl (Imjudo), and zenocutuzumab-zbco (Bizengri).

In an embodiment, the least one additional pharmaceutical agent is selected from therapies approved for lymphoma including acalabrutinib maleate monohydrate (Calquence), axicabtagene ciloleucel (Yescarta), belinostat (Beleodaq), bexarotene (Targretin), bortezomib (Velcade), brentuximab vedotin (Adcetris), brexucabtagene autoleucel (Tecartus), crizotinib (Xalkori), denileukin diftitox-cxdl (Lymphir), duvelisib (Copiktra), epcoritamab-bysp (Epkinly), glofitamab-gxbm (Columvi), ibritumomab tiuxetan (Zevalin), ibrutinib (Imbruvica), lisocabtagene maraleucel (Breyanzi), loncastuximab tesirine-lpyl (Zynlonta), mogamulizumab-kpkc (Poteligeo), mosunetuzumab-axgb (Lunsumio), nivolumab (Opdivo), obinutuzumab (Gazyva), pembrolizumab (Keytruda), pemigatinib (Pemazyre), pirtobrutinib (Jaypirca), polatuzumab vedotin-piiq (Polivy), pralatrexate (Folotyn), rituximab (Rituxan), rituximab and hyaluronidase human (Rituxan Hycela), romidepsin (Istodax), selinexor (Xpovio), siltuximab (Sylvant), tafasitamab-cxix (Monjuvi), tazemetostat hydrobromide (Tazverik), tisagenlecleucel (Kymriah), venetoclax (Venclexta), vorinostat (Zolinza), and zanubrutinib (Brukinsa).

In an embodiment, the least one additional pharmaceutical agent is selected from therapies approved for malignant mesothelioma including ipilimumab (Yervoy), nivolumab (Opdivo), and pembrolizumab (Keytruda).

In an embodiment, the least one additional pharmaceutical agent is selected from therapies approved for multiple myeloma including bortezomib (Velcade), carfilzomib (Kyprolis), ciltacabtagene autoleucel (Carvykti), daratumumab (Darzalex), daratumumab and hyaluronidase-fihj (Darzalex Faspro), elranatamab-bcmm (Elrexfio), elotuzumab (Empliciti), idecabtagene vicleucel (Abecma), isatuximab-irfc (Sarclisa), ixazomib citrate (Ninlaro), talquetamab-tgvs (Talvey), selinexor (Xpovio), and teclistamab-cqyv (Tecvayli),

In an embodiment, the least one additional pharmaceutical agent is selected from therapies approved for myelodysplastic and myeloproliferative disorders including fedratinib hydrochloride (Inrebic), imatinib mesylate (Gleevec), imetelstat sodium (Rytelo), ivosidenib (Tibsovo), momelotinib dihydrochloride monohydrate (Ojjaara), pacritinib citrate (Vonjo), pemigatinib (Pemazyre), and ruxolitinib phosphate (Jakafi).

In an embodiment, the least one additional pharmaceutical agent is selected from therapies approved for neuroblastoma including rituximab (Unituxin) and naxitamab-gqgk (Danyelza).

In an embodiment, the least one additional pharmaceutical agent is selected from therapies approved for ovarian epithelial, fallopian tube, and primary peritoneal cancers including bevacizumab (Avastin), mirvetuximab soravtansine-gynx (Elahere), niraparib tosylate monohydrate (Zejula), olaparib (Lynparza), and rucaparib camsylate (Rubraca).

In an embodiment, the least one additional pharmaceutical agent is selected from therapies approved for pancreatic cancer including belzutifan (Welireg), erlotinib hydrochloride (Tarceva), everolimus (Afinitor), olaparib (Lynparza), sunitinib malate (Sutent), and zenocutuzumab-zbco (Bizengri).

In an embodiment, the least one additional pharmaceutical agent is selected from therapies approved for plexiform neurofibroma including selumetinib sulfate (Koselugo).

In an embodiment, the least one additional pharmaceutical agent is selected from therapies approved for prostate cancer including abiraterone acetate (Zytiga), apalutamide (Erleada), bicalutamide (Casodex), cabazitaxel (Jevtana), darolutamide (Nubega), degarelix (Firmagon), enzalutamide (Xtandi), flutamide, goserelin acetate (Zoladex), leuprolide acetate (Lupron Depot, Eligard), leuprolide mesylate (Camcevi), lutetium Lu 177 vipivotide tetraxetan (Pluvicto), nilutamide (Nilandron), niraparib tosylate monohydrate and abiraterone acetate (Akeega), olaparib (Lynparza), talazoparib tosylate (Talzenna), radium 223 dichloride (Xofigo), relugolix (Orgovyx), rucaparib camsylate (Rubraca), and triptorelin pamoate (Trelstar).

In an embodiment, the least one additional pharmaceutical agent is selected from therapies approved for skin cancer including alitretinoin (Panretin), atezolizumab (Tecentriq), atezolizumab and hyaluronidase-tqjs (Tecentriq Hybreza), avelumab (Bavencio), binimetinib (Mektovi), cemiplimab-rwlc (Libtayo), cobimetinib fumarate (Cotellic), cosibelimab-ipdl (Unloxcyt), dabrafenib mesylate (Tafinlar), encorafenib (Braftovi), ipilimumab (Yervoy), nivolumab (Opdivo), nivolumab and relatlimab-rmbw (Opdualag), pembrolizumab (Keytruda), retifanlimab-dlwr (Zynyz), sonidegib (Odomzo), tebentafusp-tebn (Kimmtrak), trametinib dimethyl sulfoxide (Mekinist), vismodegib (Erivedge), and vemurafenib (Zelboraf).

In an embodiment, the least one additional pharmaceutical agent is selected from therapies approved for soft tissue sarcoma including afamitresgene autoleucel (Tecelra), alitretinoin (Panretin), atezolizumab (Tecentriq), atezolizumab and hyaluronidase-tqjs (Tecentriq Hybreza), crizotinib (Xalkori), nirogacestat hydrobromide (Ogsiveo), pazopanib hydrochloride (Votrient), sirolimus protein-bound particles (Fyarro), and tazemetostat hydrobromide (Tazverik).

In an embodiment, the least one additional pharmaceutical agent is selected from therapies approved for solid tumors anywhere in the body including dabrafenib mesylate (Tafinlar), dostarlimab-gxly (Jemperli), entrectinib (Rozlytrek), fam-trastuzumab deruxtecan-nxki (Enhertu), larotrectinib sulfate (Vitrakvi), pembrolizumab (Keytruda), repotrectinib (Augtyro), selpercatinib (Retevmo), and trametinib dimethyl sulfoxide (Mekinist).

In an embodiment, the least one additional pharmaceutical agent is selected from therapies approved for stomach (gastric) cancer including fam-trastuzumab deruxtecan-nxki (Enhertu), nivolumab (Opdivo), pembrolizumab (Keytruda), ramucirumab (Cyramza), tislelizumab-jsgr (Tevimbra), trastuzumab (Herceptin), and zolbetuximab-clzb (Vyloy).

In an embodiment, the least one additional pharmaceutical agent is selected from therapies approved for systemic mastocytosis including avapritinib (Ayvakit), imatinib mesylate (Gleevec), and midostaurin (Rydapt).

In an embodiment, the least one additional pharmaceutical agent is selected from therapies approved for thyroid cancer including cabozantinib-s-malate (Cometriq), dabrafenib mesylate (Tafinlar), lenvatinib mesylate (Lenvima), pralsetinib (Gavreto), selpercatinib (Retevmo), sorafenib tosylate (Nexavar), trametinib dimethyl sulfoxide (Mekinist), and vandetanib (Caprelsa).

In the context of combination therapy, administration of the oral non-aqueous epinephrine or epinephrine prodrug formulation of this disclosure may be simultaneous with or sequential to the administration of the one or more additional pharmaceutical agents. In another embodiment, administration of the different components of a combination therapy may be at different frequencies. The one or more additional pharmaceutical agents may be administered prior to (e.g., 5 minutes 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the oral non-aqueous epinephrine or epinephrine prodrug formulation of this disclosure.

Preferably, the administration of the oral non-aqueous epinephrine or epinephrine prodrug formulation of this disclosure in combination with one or more additional pharmaceutical agents provides a synergistic response in the subject being treated. In this context, the term “synergistic” refers to the efficacy of the combination being more effective than the additive effects of either single therapy alone. The synergistic effect of a combination therapy according to the disclosure can permit the use of lower doses and/or less frequent administration of at least one pharmaceutical agent in the combination compared to its dose and/or frequency outside of the combination. Additional beneficial effects of the combination can be manifested in the avoidance or reduction of adverse or unwanted side effects associated with the use of either therapy in the combination alone (also referred to as monotherapy).

### Methods for Treating Microbial Infection

Provided in this disclosure is a method for treating a microbial infection comprising administering a dipivefrin composition to a subject in need of treatment of a microbial infection. The microbial infection can be a bacterial, viral, fungal, or parasitic infection.

The bacterial infection can be a mycobacterial infection; a Gram positive bacterial infection, such as a Spirochete infection, Staphylococcus infection, a Streptococcus infection, a Clostridium infection, a Vibrio infection, a Bacillus infection, a Salmonella infection, a Listeria infection, or a Corynebacterium infection; or a Gram negative bacterial infection, such as an E. coli infection, a Klebsiella pneumoniae infection, an Acinetobacter baumannii infection, a Pseudomonas aeruginosa, a Neisseria gonorrhoeae infection, or a Yersinia pestis infection, Neisseria meningitides infection. A Hemophilus influenzae B, infection, a Lyme disease spirochetes infection, a Mycobacterium leprae, a Pneumococcus spp infection, a Treponema pallidum infection, a Legionella pneumophilia infection, a Brucella abortus infection, a Mycobacterium tuberculosis infection, a Mycoplasma infection, Bacillus anthracis, Streptococcus agalactiae, Streptococcus pyogenes, Escherichia coli, Neisseria gonorrhoeae, Neisseria meningitides, or a Pseudomonas aeruginosa.

The viral infection can include influenza, a herpes virus infection, a dengue virus infection, a human immunodeficiency virus infection, a hepatitis virus infection, a west Nile virus infection, a cytomegalovirus infection, a rabies virus infection, a flavivirus infection, a rhinovirus infection, a papillomavirus infection, a paramyxovirus infection, a parainfluenza virus infection, a retrovirus infection, or an infection caused by the following virus: Sendai virus, feline leukemia virus, Reo virus, polio virus, human serum parvo-like virus, simian virus 40, respiratory syncytial virus, mouse mammary tumor virus, Varicella-Zoster virus, dengue virus, rubella virus, measles virus, adenovirus, human T-cell leukemia viruses, Epstein-Barr virus, murine leukemia virus, mumps virus, vesicular stomatitis virus, Sindbis virus, lymphocytic choriomeningitis virus, or blue tongue virus.

The fungal infection can be a yeast infection or an infection by a filamentous fungus. Examples of a fungal infection include systemic candidiasis, aspergillosis, cryptococcosis, blastomycosis, coccidioidomycosis, histoplasmosis, and mucormycosis, Microsporum, Trichophyton, Epidermophyton, Sporothrix schenckii, Cryptococcus neoformans, Coccidioides immitis, Histoplasma capsulatum, Blastomyces dermatitidis, or Candida albicans.

The method for treating microbial infection can further be a combination therapy comprising administering dipivefrin and at least one or more additional chemotherapy treatment that exerts direct inhibitory effect against the microbial pathogen to the subject, herein referred to as an “antimicrobial agent.” The antimicrobial agent can be an antibiotic, an antifungal agent, an antiviral agent, an antiparasitic agent, or a combination thereof

Examples of antibiotics include a beta-lactam antibiotic, a tetracycline, a sulfonamide antibiotic, an aminoglycoside antibiotic, a macrolide antibiotic, a fluoroquinolone, and a quinolone antibiotic and a lasso peptide antibiotic. Examples of a beta-lactam antibiotic include a cephalosporin, a penicillin, a monobactam, a carbapenem, and a carbacephem. Examples of aminoglycoside antibiotic include streptomycin, dihydrostreptomycin, amikacin, apramycin, arbekacin, astromicin, bekanamycin, dibekacin, framycetin, gentamicin, hygromycin B, isepamicin, kanamycin, neomycin, netilmicin, paromomycin, rhodostreptomycin, ribostamycin, sisomycin, spectinomycin, tobramycin, and verdamicin. Examples of a fluoroquinolone include ciprofloxacin, clinafloxacin, enoxacin, fleroxacin, gatifloxacin, moxifloxacin, gemifloxacin, grepafloxacin, levofloxacin, norfloxacin, sparfloxacin, and trovafloxacin. Examples of a quinolone include cinoxacin, garenoxacin, and nalidixic acid. Examples of a macrolide include azithromycin, clarithromycin, dirithromycin, erythromycin, lincomycin, roxithromycin, troleandomycin, telithromycin, and spectinomycin. Examples of a tetracycline include demeclocycline, doxycycline, minocycline, oxytetracycline, tgecycline, and tetracycline. Examples of a sulfonamide antibiotic include Sulfamethizole, Sulfamethoxazole, Sulfisoxazole, Trimethoprim-Sulfamethoxazole. Examples of lasso peptide includes lariocidin. Examples of antibiotics also include Zevtera (ceftobiprole medocaril sodium), Blujepa (gepotidacin), Emblaveo (aztreonam/avibactam), Pivya (pivmecillinam), Cefiderocol, Lefamulin, Imipenem/cilastatin/relebactam, Pretomanid, and Tebipenem HBr.

Examples of an antifungal agent include isavuconazole, rezafungin, and ibrexafungerp, Amphotericin B, Candicidin, Filipin, Hamycin, Natamycin, Nystatin, Rimocidin, Bifonazole, Butoconazole, Clotrimazole, Econazole, Fenticonazole, Fosmanogepix, Isoconazole, Ketoconazole, Luliconazole, Miconazole, Olorofim, Omoconazole, Oteseconazole, Oxiconazole, Sertaconazole, Sulconazole, Tioconazole, Albaconazole, efinaconazole, epoxiconazole, fluconazole, isavuconazole, itraconazole, posaconazole, propiconazole, ravuconazole, terconazole, voriconazole, abafungin, allylamines, Anidulafungin, Caspofungin, Micafungin, Aurones, Benzoic acid, Ciclopirox, Flucytosine, Griseofulvin, Haloprogin, Tolnaftate, undecylenic acid, Crystal violet, and Balsam of Peru.

Examples of an antiviral agent useful in treating viral infections such as influenza include neuraminidase inhibitors (e.g., oseltamivir and zanamivir) and M2 ion channel inhibitors (e.g., amantadine and rimantadine) and agents that inhibit viral replication, transcription, reverse transcription, or viral particle production. In one embodiment, the antiviral agent is selected from the group consisting (+)-Calanolide A; (+)-Dihydrocalanolide A; 2′-C-methyl-7-deazaadenosine; 2′-C-Methylcytidine; 2-Nor-cyclic GMP; 3,4-Dicaffeoylquinic acid; 3-Hydroxymethyl dicamphanoyl khellactone; 3-Hydroxyphthaloyl-beta-lactoglobulin; 3-Nitrosobenzamide; 4-Azidothymidine; 4-Methyl dicamphanoyl khellactone; 739W94; Abacavir; AC 2; Acemannanl Acetylcysteine-Zambon; Aciclovir (e.g., extended release, controlled release, topical patch); Aciclovir-PMPA; ACP HIP; Actinohivin; Adamantylamide dipeptide; Adefovir (e.g., dipivoxil); Afovirsen; Aidfarel; AL 721; Alamifovir; Albuferon; Albumin/interferon-alpha; Aldesleukin; Alovudine; Alpha HGA; Alpha-IPDX; Alpha-antitrypsin; Alvircept sudotox; Alvocidib; Amantadine; Amdoxovir; Amidinomycin; Aminopeptidase; Amitivir; Ampligen; Amprenavir; Ancriviroc; Andrographis; Anti-CCR5 monoclonal antibody; Anti-CCR5/CXCR4 sheep monoclonal antibody; Anti-CD3 monoclonal antibody CD4IgG conjugate; Anti-CD4 monoclonal antibody; Anti-CD7 monoclonal antibody; Anti-CD8 monoclonal antibody; Anti-CMV monoclonal antibody; Anti-hepatitis B ribozyme; Anti-HIV catalytic antibody; Anti-HIV immunotoxin (IVAX); Anti-HIV-1 human monoclonal; antibody 2FS; Anti-HIV-1 human monoclonal; antibody 2G12; Anti-HIV-1 human monoclonal; antibody 4E10; Antineoplaston AS2 1 (e.g., oral); Anti-RSV antibody (Intracel, Corp.); Antisense oligonucleotide PB2; Aop-RANTES; Aphidicolin; Aplaviroc; Apricitabine; Arbidol; Artemether; Atermisinin; Artesunate; Atazanavir; Atevirdine; Atorvastatin; Avarol; Azidodideoxyguanosine; Azodicarbonamide; Bafilomycin A1; Baicalin; Bavituximab; Bellenamine; Benanomicin A; Benzalkonium (e.g., chloride); Benzalkonium chloride/octoxynol 9 (e.g., vaginal gel); Beta-D-FDOC; Beta-L-ddC; Beta-L-FddC; Bevirimat; Boceprevir (SCH 503034); Brecanavir; Brefeldin A; Brequinar; Brivudine; Calcium spirulan; Canventol; Capravirine; Carbendazim; Carbocyclic deazaadenosine; Carbopol polymer gel; Carbovir; CD4 fusion toxin; CD4 IgG; CD4-ricin chain A; Celgosivir; CellCept; Cellulose sulfate; Cepharanthine; Ceplene; Chloroquine (e.g., phosphate); Cidofovir; Ciluprevir (BILN 2061); Civacir; Civamide; Clevudine; Cobra venom peptide; Colloidal silver; Conocurvone; Cosalane; Costatolide; Crofelemer; Curcumin; Curdlan sulfate; Cyanovirin-N; Cyclosporine; Cytarabine; Cytomegalovirus immune globulin; DAB486interleukin-2; Dacopafant; Dapivirine; Darunavir; D-aspartic-beta-hydroxamate; Deazaadenosine; Deazzaneplanocin A; Delavirdine; Delmitide; Denileukin diftitox; Deoxyfluoroguanosine; Dexelvucitabine; Dextran sulfate; Dextrin 2-sulfate; Didanosine; Dideoxyadenosine; Dideoxyguanosine; Dideoxythymidine; Didox; Dihydroartemisinin; Dihydrocostatolide; Dinitrochlorobenzene; Docosanol; Droxinavir; EB-Foscarnet; Edodekin alfa; Edoxudine; Efavirenz; EGS 21; EHC 18; EHT 899; Elvucitabine; EM 1421; EM 2487; Emivirine; Emtricitabine; Emtricitabine/tenofovir disoproxil fumarate; Enfuvirtide; Entecavir; Eosinophil-derived neturalizing agent; Episiastatin B; Etanercept; Ether lipid analogue; Etoviram; Etravirine; Famciclovir; Fas-ligand inhibitor; Fasudil; Fattiviracin A1; Feglymycin; Felvizumab; Fiacitabine; Fialuridine; Floxuridine; Flutimide; Fluvastatin (e.g., sodium); Fornivirsen; Fosalvudine tidoxil; Fosamprenavir; Foscarnet Sodium; Fozivudine; Galactan sulfate; Ganciclovir; Gemcitabine (e.g., hydrochloride); Ginseng; Glamolec; Glutathionarsenoxide; Glycovir; Glycyrrhizin; Gossypol; Hepatitis B immune globulin; Hepatitis C immune globulin; Hepex C; HEPT; Heptazyme; Histamine; Histamine dihydrochloride (e.g., injection, oral); HIV DNA vaccine (Antigen Express, Inc.); HIV immune globulin; HIV immune plasma; HuMax-HepC; Hydroxycarbamide; Hydroxychloroquine; Hypericin; Idoxuridine; Imiquimod; ImmStat; ImmuDyn; Immunocal; Imreg 1; Incadronic acid; Indinavir; Infliximab; Influenza matrix protein Zn2+ finger peptide; Ingenol Triacetate; Inophyllum B; Inosine pranohex; Interferon; Interferon Alfa-2a; Interferon alfa-2b (e.g., inhalation); Interferon alfacon-1; Interferon alpha (e.g., sustained release, intranasal, Omniferon); Interferon alpha-2b (e.g., controlled release or tranadermal); Interferon alpha-2b gene therapy; Interferon alpha-n3; Interferon beta-1a; Interferon beta-1b; Interferon gamma-1b; Interferon omega; Interferon-tau; Interleukin 10 (e.g., human recombinant); Interleukin-1 receptor type I; Interleukin-13; Interleukin-15; Interleukin-16; Interleukin-2 agonist; Interleukin-4; IPdR: Ipilimumab; Isatoribine; Kamizol kethoxal; Kijimicin; Kistamicin; Kootikuppala; L HAS ara AMP; Lactoferrin; Lamivudine; Lamivudine/abacavir; Lamivudine/zidovudine; Lamivudine/zidovudine/abacavir; Lasinavir; L-chicoric acid; Lecithinized superoxide dismutase; Leflunomide; Lentinan; Leukocyte interleukin injection (CEL-SCI Corp.); Leukotriene B4-LTB4; Levcycloserine; Levofloxacin; Lexithromycin; Licorice root; Liposomal ODG-PFA-OMe; Lithium succinate; Lobucavir; Lodenosine; Lopinavir; Lovastatin; Loviride; Lufironil; Lysozyme; Madu; Mannan sulfate; MAP 30; Maraviroe; Maribavir; Masoprocol; MB-Focarnet; Medusa Interferon; Merimepodib (VX-497); Met-enkephalin; Methisazone; Mevastatin; Michellamine B; Miglustat; Milk thistle; Mitoquinone; Mivotilate; Monoclonal antibody 1F7; Monoclonal antibody 2F5; Monoclonal antibody 3F12; Monoclonal antibody 447-52D; Monoclonal antibody 50-61A; Monoclonal antibody B4; Monoclonal antibody HNK20; Monoclonal antibody NMO1; Mopyridone; Moroxydine; Motavizumab; Motexafin gadolinium; Mozenavir; Murabutide; Mycophenolate mofetil; Mycophenotic Acid; Navuridine; Nalfinavir (e.g., mesylate); Neomycin B-arginine conjugate; Neotripterifordin; Nevirapine; Nitazoxanide; Nitric oxide (e.g., ProStrakan); Nitrodeazauridine; Nonakine; Octoxynol 9; Oltipraz; OMaciclovir; Opaviraline; Oragen; Oseltamivir; Oxetanocin; Oxothiazolidine carboxylate; Palinavir; Palivizumab; Papuamide A; Pegaldesleukin; Peginterferon alfa-2a; Peginterferon alfa-2b; PEGinterferon alfacon-1; PEGylated interferon; Pegylated thymalfasin; Peldesine; Penciclovir; Pentosan polysulfate; Pentoxifylline; Peptide T; Peramivir; Phellodendrine; Phosphatidyllamivudine; Phosphatidylzalcitabine; Phosphatidylzidovudine; Phosphazid; Phosphinic cyclocreatine; Pinosylvin; Pirodavir; Pleconaril; Plerixafor; podophylltoxin; Poly ICLC; Polyadenylic polyuridylic acid; Polysaccharide K; Pradefovir; Pradimicin A; Prasterone; Probucol (Vyrex Corp.); Propagermanium; Prostratin; Pseudohypericin; Pyriferone; Quinbene; Quinxapeptin A; Quinoxapeptin B; Raluridine; Ramatroban; Ranpirnase; Regavirumab; Resiquimod; Resobene; Respiratory syncytial virus immune globulin; Retrogen; Ribavirin; Rilpivirine; Rimantadine; Ritonavir; Robustaflavone; Rolipram; Rosiglitazone; Rubitecan; Rupintrivir; Saquinavir (e.g., mesylate); Sargramostim; Schisandra; Semapimod; Sevirumab; Siamycin 1; Siamycin 2; sICAM-1; Sifuvirtide; Silipide, Simvastatin; Simvastatin hydroxyl acid, ammonium salt; Sizofiran; Sodium laurilsulfate; Solutein; Sorivudine (e.g., tropical); Sparfosic acid; SpecifEx-Hep B; Stachyflin stallimycin; Stampidine; Statolon; Stavudine; Steponin; Suksdorfin; Sulfated maltoheptaose; Superoxide dismutase; Suramin (e.g., sodium); Talviraline; TAP 29; Taribavarin; TASP; Tecleukin; Tecogalan (e.g., sodium); TEI 2306; Telaprevir (VX-950); Telbivudine; Telinavir; Temacrazine; Tenidap; Tenofovir; Tenofovir disoproxil fumarate; Thalidomide; Thiophosphonoformic acid; Thiovir; Thymalfasin (e.g., Zadaxin); Thymoctonan; Thymosin fraction 5; Thymotrinan; Thymus extract; Tifuvirtide; Tilarginine; Tipranavir; Tiviciclovir; Tivirapine; TNF-alpha inhibitor; Todoxin; TOFA; Tomeglovir; Transforming growth factor-alpha; TraT; Trecovirsen; Tremacamra; Trichosanthin; Triciribine; Triconal; Trifluridine; Trimidox; Trodusquemine; Tromantadine; Trovirdine; Tucaresol; Tunicamycin; Tuvirumab; Ubenimex; Ukrain; Ursodeoxycholic acid; valaciclovir; valganciclovir; valopicitabine; valtorcitabine; Varicella zoster immune globulin; Vesnarinone; Vicriviroc; vidarabine; vincristine (e.g., sulfate); viraprexin; virodene; Virostat; Viscum album extract; Zalcitabine; Zanamivir; Zidovudine (e.g., phosphate-didanosine dimer); Zidovudine triphosphate mimics; and combinations thereof.

In one embodiment, the antiviral agent is selected from the group consisting Abacavir, Acyclovir (Aciclovir), Adefovir, Amantadine, Amprenavir (Agenerase), Ampligen, Arbidol, Atazanavir, Atripla, baloxavir marboxil, cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fixed dose combination (antiretroviral), Fomivirsen, Fosamprenavir, Foscarnet, Fosfonet, Fusion inhibitor, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nitazoxanide, Nucleoside analogues, Novir, Oseltamivir, Peginterferon alfa-2a, Penciclovir, Peramivir, Pleconaril, Podophyllotoxin, Protease inhibitor, Raltegravir, Reverse transcriptase inhibitor, Ribavirin, Rimantadine, Ritonavir, Pyramidine, Saquinavir, Sofosbuvir, Stavudine, Synergistic enhancer (antiretroviral), Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), Zidovudine and combinations thereof.

Examples of anti-parasitic agents include Abametapir, Bephenium, Diethylcarbamazine, Ivermectin, Moxidectin, Niclosamide, Piperazine, Praziquantel, Pyrantel, Pyrvinium, Albendazole, Flubendazole, Mebendazole, Nitazoxanide, Triclabendazole, Thiabendazole, Benzyl benzoate, O-hydroxybutyrate, Benzyle benzoate/disulfiram, Lindane, Malathion, Permethrin, Benzyl alcohol, Piperonyl butoxide/pyrethrins, Spinosad, and Crotamiton.

### Liquid Pharmaceutical Formulations

The disclosure provides non-aqueous liquid pharmaceutical formulations comprising epinephrine or an epinephrine prodrug, such as dipivefrin (e.g. L-dipivefrin), dipivefrin hydrochloride (e.g. L-dipivefrin hydrochloride) as a pharmaceutical agent. The formulations contain about 1 mg/mL, 5 mg/mL, 10 mg/mL, 15 mg/mL, 20 mg/mL, 25 mg/mL, 30 mg/mL, 35 mg/mL, 45 mg/mL, 50 mg/mL, 55 mg/mL, 60 mg/mL, 65 mg/mL, 70 mg/mL, 75 mg/mL, 80 mg/mL, 85 mg/mL, 95 mg/mL, 100 mg/mL, 105 mg/mL, 110 mg/mL, 115 mg/mL, 120 mg/mL, 125 mg/mL, 130 mg/mL, 135 mg/mL, 140 mg/mL, 145 mg/mL, 150 mg/mL, 155 mg/mL, 160 mg/mL, 165 mg/mL, 170 mg/mL, 175 mg/mL, 180 mg/mL, 185 mg/mL, 190 mg/mL, 195 mg/mL, 200 mg/mL, 210 mg/mL, 220 mg/mL, 230 mg/mL, 240 mg/mL, 250 mg/mL, 260 mg/mL, 270 mg/mL, 280 mg/mL, 290 mg/mL or 300 mg/mL of the pharmaceutical agent, e.g. dipivefrin hydrochloride, where the weight is based on the weight of dipivefrin free base or the weight of dipivefrin hydrochloride as specified, i.e. 10 mg/mL dipivefrin contains 10 mg/mL dipivefrin or 11.03 mg/mL dipivefrin hydrochloride. In the examples, weight of dipivefrin hydrochloride means the weight of the hydrochloride salt and is not based on the free base weight. In some embodiments the formulations of this disclosure contain from about 1 mg/mL to 300 mg/mL, 1 mg/mL to 150 mg/mL, 1 mg/mL to 100 mg/mL, 1 mg/mL to 50 mg/mL, 5 mg/mL to 200 mg/mL, 5 mg/mL to 150 mg/mL, 5 mg/mL to 100 mg/mL, 5 mg/mL to 50 mg/mL, 10 mg/mL to 200 mg/mL, 10 mg/mL to 150 mg/mL, 10 mg/mL to 100 mg/mL, or 10 mg/mL to 50 mg/mL of the pharmaceutical agent, e.g. dipivefrin hydrochloride. In some embodiments the formulation is prepared so that a unit dosage amount of the formulations contains about 0.025 mg, 0.05 mg, 0.1 mg, 0.5 mg, 1 mg, 2 mg, 3 mg, 4 mg, 5 mg, 6 mg, 7 mg, 8 mg, 10 mg, 11 mg, 12 mg, 13 mg, 14 mg, 15 mg, 16 mg, 17 mg, 18 mg, 19 mg, 20 mg, 21 mg, 22 mg, 23, mg, 24 mg, 25 mg, 26 mg, 27 mg, 28 mg, 29 mg, or 30 mg of the pharmaceutical agent, e.g. dipivefrin hydrochloride. In some embodiments the dosage form provides about 0.5 mg to 30 mg, 1 mg to 20 mg, 2 mg to 20 mg, 5 mg to 20 mg, 0.5 mg to 15 mg, 1 mg to 15 mg, 2 mg to 15 mg, 5 mg to 15 mg, 0.5 mg to 10 mg, 1 mg to 10 mg, 2 mg to 10 mg, or 5 mg to 10 mg of the pharmaceutical agent, e.g. dipivefrin hydrochloride.

The formulation can provide about 0.001 mg/kg to about 1000 mg/kg, about 0.01 mg/kg to about 100 mg/kg, about 10 mg/kg to about 250 mg/kg, about 0.1 mg/kg to about 15 mg/kg; or any range in which the low end of the range is any amount between 0.001 mg/g and 900 mg/kg and the upper end of the range is any amount between 0.1 mg/kg and 1000 mg/kg (e.g., 0.005 mg/kg and 200 mg/kg, 0.05 mg/kg and 20 mg/kg) of the pharmaceutical agent, e.g. dipivefrin hydrochloride.

The non-aqueous epinephrine or epinephrine prodrug formulation of this disclosure includes a solvent. It can contain just one solvent, selected from a first non-aqueous solvent and a second non-aqueous solvent. The non-aqueous epinephrine or epinephrine prodrug formulation of this disclosure usually contains a first non-aqueous solvent and a second non-aqueous solvent. The non-aqueous epinephrine or epinephrine prodrug formulation of this disclosure includes at least 10%, (w/v) 10%, at least 12%, at least 14%, at least 16%, at least 18%, at least 20%, at least 22%, at least 24%, at least 26%, at least 28%, at least 30%, at least 32%, at least 34%, at least 36%, at least 38%, at least 40%, at least 42%, at least 44%, at least 46%, at least 48%, or at least 50%, at least 52%, at least 54%, at least 56%, at least 58%, at least 60%, at least 62%, at least 64%, at least 66%, at least 68%, at least 70%, at least 82%, at least 84%, at least 86%, at least 88%, or at least 90%, of a first non-aqueous solvent. The non-aqueous formulation epinephrine or epinephrine prodrug formulation can include (w/v) 10%-90%, 20%-90%, 25%-90%, 30%-90%, 35%-90%, 40%-90%, 45%-90%, 10%-80%, 20%-80%, 25%-80%, 30%-80%, 35%-80%, 40%-80%, 45%-80%, 10%-75%, 20%-75%, 25%-75%, 30%-75%, 35%-75%, 40%-75%, 45%-75%, 10%-70%, 20%-70%, 25%-70%, 30%-70%, 35%-70%, 40%-70%, 45%-70%, 10%-65%, 20%-65%, 25%-65%, 30%-65%, 35%-65%, 40%-65%, 45%-65%, 10%-60%, 20%-60%, 25%-60%, 30%-60%, 35%-60%, 40%-60%, or 45%-60% of a first non-aqueous solvent. In certain circumstances, the non-aqueous epinephrine or epinephrine prodrug formulation of this disclosure includes 100% of a first non-aqueous solvent. In an embodiment the first non-aqueous solvent is dehydrated ethyl alcohol.

The non-aqueous formulation epinephrine or epinephrine prodrug formulation of this disclosure includes a second non-aqueous solvent. The non-aqueous formulation epinephrine or epinephrine prodrug formulation of this disclosure includes at least 20%, at least 22%, at least 24%, at least 26%, at least 28%, at least 30%, at least 32%, at least 34%, at least 36%, at least 38%, at least 40%, at least 42%, at least 44%, at least 46%, at least 48%, or at least 50%, at least 52%, at least 54%, at least 56%, at least 58%, at least 60%, at least 62%, at least 64%, at least 66%, at least 68%, at least 70%, at least 82%, at least 84%, at least 86%, at least 88%, or at least 90% of a second non-aqueous solvent or co-solvent. The non-aqueous formulation epinephrine or epinephrine prodrug formulation can include 10%-90%, 20%-90%, 25%-90%, 30%-90%, 35%-90%, 40%-90%, 45%-90%, 10%-80%, 20%-80%, 25%-80%, 30%-80%, 35%-80%, 40%-80%, 45%-80%, 10%-75%, 20%-75%, 25%-75%, 30%-75%, 35%-75%, 40%-75%, 45%-75%, 10%-70%, 20%-70%, 25%-70%, 30%-70%, 35%-70%, 40%-70%, 45%-70%, 10%-65%, 20%-65%, 25%-65%, 30%-65%, 35%-65%, 40%-65%, 45%-65%, 10%-60%, 20%-60%, 25%-60%, 30%-60%, 35%-60%, 40%-60%, or 45%-60% of a second non-aqueous solvent. In certain circumstances, the non-aqueous epinephrine or epinephrine prodrug formulation of this disclosure includes 100% of a second non-aqueous solvent. In an embodiment the second non-aqueous solvent is propylene glycol, polyethylene glycol, 1,2-propanediol, 1,3-propanediol, or glycerol or Transcutol® (diethylene glycol monoethyl ether, DEGEE), a combination of the foregoing can comprise the second non-aqueous solvent.

The non-aqueous epinephrine or epinephrine prodrug formulation of this disclosure can also include a terpene or terpenoid. Terpenes are a class of compounds, primarily derived from plants having the isoprene unit (C₅H₈), a 5-carbon compound in their backbones. Terpenoids, also known as isoprenoids, are a class of naturally occurring organic chemicals derived from the 5-carbon compound isoprene and are derivatives of terpenes, diterpenes, etc. While sometimes used interchangeably with “terpenes”, terpenoids contain additional functional groups, usually containing oxygen. Terpenes and terpenoids are used interchangeably in this disclosure. The terpene can be menthol, e.g. L-Menthol. Terpenes suitable for use in the disclosed formulations include L-Menthol, Eucalyptol, and Eugenol. In other embodiments the terpene is selected from menthol (e.g., L-Menthol), Menthone, Eucalyptol, Eugenol, Terpineol, Linalool, Limonene (e.g., D-Liminene), Myrcene, 1,8-Cineole, 1,4-Cineole, Anethole, α-Bisabolol, Borneol, Camphor, Carvacrol, Carvone, Cymene, Farnesol, Fenchone, Geraniol, Nerolidol, α-Pinene oxide, Pulegone, Rose oxide, Safranal, Terpinen-4-ol, α-Terpineol, Tetrhydrogeraniol, Valencene, Vervenone. The terpene can be present at a concentration of 0.005 to 0.25 g/mL, 0.005 to 0.2 g/mL, 0.005 to 0.15 g/mL, 0.005 to 0.1 g/mL, 0.005 to 0.05 g/mL, 0.01 to 0.25 g/mL, 0.01 to 0.2 g/mL, 0.01 to 0.15 g/mL, 0.01 to 0.1 g/mL, 0.01 to 0.05 g/mL, 0.05 to 0.25 g/mL, 0.05 to 0.2 g/mL, 0.05 to 0.15 g/mL, or 0.05 to 0.1 g/mL

The non-aqueous epinephrine or epinephrine prodrug formulations of this disclosure can also include an essential oil. Essential oils are natural products with a complex composition that are distilled/extracted from plants. Terpenes are the most common class of chemical compounds present in essential oils. Essential oils suitable for use in the disclosed formulations include clove oil, peppermint oil, lemon oil, Eucalyptus oil, eucalyptol, copaiba oil, cinnamon oil, tea tree oil, aloe vera oil, cumin oil, rose oil, Angelica oil, chuanxiong oil, Cyperus oil, turpentine oil. In certain circumstances, mint flavors which contain terpenes can be used.

In addition to a first non-aqueous solvent a second non-aqueous solvent, the formulation can contain a surfactant, flavoring agent, sweetening agent, an antifoaming agent, antioxidant, preservative, coloring agent, a viscosity modifying agent, permeation enhancer, or mucoadhesive substance.

The surfactant can be a surfactant selected from a lipid, such as Labrasol®, sodium deoxycholate, a caprylocaproyl macrogol-8 glyceride, a caprylocaproyl polyoxyl-8 glyceride, a polyoxylglyceride, a polyoxyethylene stearate, a sorbitan fatty acid ester, and a poloxamer, an alkyl glycoside, a bile salt and its derivatives (such as sodium glycocholate, sodium taurocholate, and sodium taurodihydrofusidate), a phospholipid, or a combination of any of the foregoing. In some embodiments, the alkyglycoside has an alkyl chain including between 8 to 20 carbons. In some embodiments, the alkyglycoside is selected from the group consisting of undecyl maltoside, dodecyl maltoside, tridecyl maltoside, tetradecyl maltoside, sucrose mono-dodecanoate, sucrose mono-tridecanoate, and sucrose mono-tetradecanoate. In some embodiments, the alkylglycoside is dodecyl beta-D-maltoside (DDM). The surfactant can be an anionic surfactant, such as sodium stearate, sodium dodecyl sulfate (SDS), alkylpolyoxyethylene sulfate, alkylbenzene sulfonate, sodium dodecyl benzene sulfonate, or sodium cholate. The surfactant can be a cationic surfactant such as hexadecyltrimethylammonium bromide, benzalkonium chloride, sodium lauryl sulfate, cetrimide, sorbitan monopalmitate, dodecyl pyridinium chloride. The surfactant can be a nonionic surfactant such as heptaoxyethylene monohexadecylether. The surfactant can be an ampholytic (Zitterionic) surfactant such as lecithin or N-dodecyl alanine. The surfactant can be present at a concentration of 0.001 to 10%, 0.05 to 5%, 0.05 to 3%, 0.5 to 2.5% 0.5 to 2%, 0.1 to 5%, 0.1 to 4%, 0.1 to 3%, 0.1 to 2.5% or 0.1 to 2%.

The formulation can contain a flavoring agent or sweetening agent. “Flavoring agents” and/or “sweeteners” useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, dextrose, Eucalyptus, eugenol, fructose, fruit punch, ginger, glycyrrhetinate, Glycyrrhiza (licorice) syrup, grape, grapefruit, honey, isomalt, lemon, lime, lemon cream, monoammonium glyrrhizinate (MagnaSweet®), maltol, mannitol, maple marshmallow, menthol. Mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, Stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti frutti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Antifoaming agents” reduce foaming during processing which can result in coagulation of aqueous dispersions, bubbles in the finished film, or impair processing. Exemplary anti-foaming agents include silicon emulsions or sorbitan sesquioleate.

Colorants or coloring agents are used to impart a distinctive appearance to the pharmaceutical dosage forms. Suitable coloring agents include food, drug, and cosmetic colors (FD&C), drug and cosmetic colors (D&C), or external drug and cosmetic colors (Ext. D&C). These colors are dyes, their corresponding lakes, and certain natural and derived colorants. Lakes are dyes absorbed on aluminum hydroxide. Some examples of widely used colorants in pharmaceuticals include FD&C Blue No. 1-Brilliant Blue (blue shade), FD&C Blue No. 2-Indigotine, (indigo shade), FD&C Red No. 3-Erythrosine, (pink shade), FD&C Red No. 40-Allura Red, (red shade), FD&C Yellow No. 5-Tartrazine, (yellow shade) and FD&C Yellow No. 6-Sunset yellow, (orange shade).

Other examples of coloring agents include known azo dyes, organic or inorganic pigments, or coloring agents of natural origin. Inorganic pigments are preferred, such as the oxides or iron or titanium, these oxides, being added in concentrations ranging from about 0.001 to about 10%, and preferably about 0.5 to about 3%, including greater than 0.001%, greater than 0.01%, greater than 0.1%, greater than 0.5%, greater than 1%, greater than 2%, greater than 5%, about 10%, greater than 10%, less than 10%, less than 5%, less than 2%, less than 1%, less than 0.5%, less than 0.1%, less than 0.01%, or less than 0.001%, based on the weight of all the components.

“Antioxidants” include, for example, butylated hydroxytoluene (BHT), sodium ascorbate, ascorbic acid, sodium metabisulfite and tocopherol. In certain embodiments, antioxidants enhance chemical stability where required. In addition to antioxidants, the formulation can contain another stabilizing agent such as a metal chelating agent, thiol containing compound, and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosane polysulfate and other heparinoids, (m) cyclodextrins, (l) pentosan polysulfate and other heparinoids, (m) divalent cations such as magnesium and zinc; or (n) combinations thereof.

In some embodiments, antioxidation can be achieved by physical exclusion of oxygen via inert gas flushing such as nitrogen or argon flushing prior to packaging of the non-aqueous epinephrine or epinephrine prodrug formulation of this disclosure in a suitable container/closure system.

In some embodiments, compositions provided herein also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide, cetylpyridinium chloride and the parabens.

In some embodiments, the pharmaceutical combination and/or composition described herein also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases, and buffers are included in an amount required to maintain pH of the composition in an acceptable range.

In some embodiments, the pharmaceutical combination and/or compositions also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate, or bisulfite anions; suitable salts include sodium chloride, potassium chloride, sodium thiosulfate, sodium bisulfite and ammonium sulfate.

In some embodiments, the pharmaceutical combination and/or compositions also include one or more mucoadhesive substances/polymers that prolong the retention of the pharmaceutical agent at the site of administration. Polymers with carboxyl, amine, or hydroxyl groups and a certain molecular weight can help prolong the retention of a system with the mucous layer. Examples of mucoadhesive substances/polymers are chitosan and modified chitosan, poly(acrylic acid) and its derivatives, alginates, pectin, poly(methacrylic acid), sodium carboxymethyl cellulose, hyaluronic acid, hyaluronate sodium, hydroxyl propyl cellulose (HPC), hydroxyl propyl methyl cellulose (HPMC), hydroxyl ethyl cellulose (HEC), carboxy methyl cellulose (CMC).

In some embodiments, the epinephrine or epinephrine prodrug compositions of this disclosure also include at least one permeation enhancer that safely and reversibly alters the permeability restrictions of the oral mucosa. Examples of permeation enhancers are bile salts (both natural and synthetic), surfactants, fatty acids and their derivatives, chelators, cyclodextrins, chitosan, Azone® (laurocapram), phytoextract, Transcutol® (a purified form of diethylene glycol monoethyl ether (DEGEE)), and sulfoxides. Examples of bile salts include sodium glycodeoxycholate (SGDC) and sodium taurodeoxycholate (TDC) and tri-hydroxy bile salts, sodium glycocholate (GC) and sodium taurocholate (TC), sodium fusidate; glycocholate or deoxycholate and their salts; Examples of fatty acids and derivatives include sodium laurate, oleic acid, oleyl alcohol, monoolein, and palmitoylcarnitine; Chelators include disodium EDTA, sodium citrate. Surfactants include alkyl glycosides such as dodecyl beta-D-maltoside (DDM), sodium laurylsulfate, Azone®, sodium cholate, sodium 5-methoxysalicylate, sorbitan laurate, glyceryl monolaurate, octoxynonyl-9, laureth-9, polysorbates, sterols, or glycerides, such as captylocaproyl polyoxylglycerides, e.g., Labrasol. A phytoextract can be an essential oil or composition including essential oils extracted by distillation of the plant material. In certain circumstances, the phytoextract can include synthetic analogues of the compounds extracted from the plant material (i.e., compounds made by organic synthesis). The phytoextract can include a phenylpropanoid, for example, phenyl alanine, eugenol, eugenol acetate, a cinnamic acid, a cinnamic acid ester, a cinnamic aldehyde, a hydrocinnamic acid, chavicol, or safrole, or a combination thereof. The phytoextract can be an essential oil extract of a clove plant, for example, from the leaf, stem, or flower bud of a clove plant. The clove plant can be Syzygium aromaticum. The phytoextract can include 20-95% eugenol, including 40-95% eugenol, including 60-95% eugenol, and for example, 80-95% eugenol. The extract can also include 5% to 15% eugenol acetate. The extract can also include caryophyllene. The extract can also include up to 2.1% a-humulene. Other volatile compounds included in lower concentrations in clove essential oil can be pinene, limonene, farnesol, benzaldehyde, 2-heptanone and ethyl hexanoate.

“Dispersing agents,” and/or “viscosity modulating agents” include material that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (s630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as e.g., gum tragacanth and gum acacia, guar gum, xanthans, including xanthan gum, sugars, cellulosics, such as, e.g., sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate. “Viscosity enhancing agents” also include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

The term “diluent” refers to chemical compounds that are used to dilute the compound of interest prior to delivery. Diluents can be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar’; monobasic calcium sulfate monohydrate, calcium sulfate dehydrate; calcium sulfate trihydrate, dextrates; hydrolyzed cereal solids, amylose; powdered cellulose, calcium carbonate; glycine, kaolin; mannitol, sodium chloride; inositol, bentonite, and the like.

The non-aqueous epinephrine or epinephrine prodrug formulation of this disclosure, e.g. a dipivefrin hydrochloride formulation, may be used in any of the methods disclosed herein.

The non-aqueous epinephrine or epinephrine prodrug formulation of this disclosure can be prepared by a process comprising combining the dipivefrin with a pharmaceutically acceptable excipient. Thus, the disclosure further encompasses the use of the above-described dipivefrin composition in the manufacture of a pharmaceutical composition. Excipients may be added to facilitate manufacture, enhance stability, adjust pH, adjust isotonicity, enhance absorption, enhance product characteristics, enhance bioavailability, enhance patient acceptability, etc.

The non-aqueous epinephrine or epinephrine prodrug formulation of this disclosure may be packaged in a container closure system under an inert atmosphere such as nitrogen or argon to further enhance stability.

Also disclosed is a combination pharmaceutical composition comprising epinephrine or an epinephrine prodrug, e.g dipivefrin hydrochloride, as a first pharmaceutical agent and at least one other pharmaceutical agent and at least one pharmaceutical excipient.

The term “combination” as used herein, means a product that results from the mixing or combining of dipivefrin and any additional therapeutic agents and includes both fixed and non-fixed combinations. The term “fixed combination” means that dipivefrin and the additional therapeutic agents are administered in a single entity or dosage form. The term “non-fixed combination” means that dipivefrin and the additional therapeutic agents are administered as separate entities or dosage forms either simultaneously, concurrently or sequentially with no specific intervening time limits, wherein such administration provides therapeutically effective levels of the two compounds in the body of the patient. The latter also applies to cocktail therapy, e.g. the administration of three or more active ingredients.

It should be understood that in addition to the ingredients particularly mentioned above, the compounds and compositions described herein may include other agents conventional in the art having regard to the type of formulation in question, for example those suitable for oral administration may include sweetening agents, flavoring agents, coloring agents, taste masking agents, and preserving agents in order to provide pharmaceutically elegant and palatable preparations.

### Drug Delivery Devices and Kits

The epinephrine and epinephrine prodrug formulations of this disclosure are preferably delivered via a spray device (e.g. a nasal spray device or a sublingual spray device or a throat spray device or a topical spray device). Also provided in this disclosure are drug delivery devices comprising a formulation described herein. Metered spray pumps have dominated the spray drug delivery market since they were introduced. The pumps typically deliver 100 μL (25-250 μL) per spray, and they offer high reproducibility of the emitted dose and plume geometry in in vitro tests. In certain embodiments, the spray device is pre-primed. In certain embodiments, the spray device can be primed before use. In certain embodiments, the spray device can be actuated with one hand. Examples of standard metered spray pumps include those offered by Aptar Pharma, Inc., such as the multi-dose“classic technology platform” spray devices, and by BD Medical Pharmaceutical Systems, such as the Accuspray™ system. Such devices comprise a reservoir which holds multiple doses of the spray formulation (e. g., 50, 60, 100, 120, 150, or 200 doses), a closure (e. g., screw, crimp, or snap-on), and an actuator which delivers anywhere from 45 to 1000 μL (e. g., 50, 100, 140, 150, or 200 μL) of fluid per actuation to comprise a single dose. The actuator may be configured to count doses, deliver gel formulations, deliver in an upside-down configuration, etc.

In traditional multi-use spray pump systems, antimicrobial preservatives are typically required to maintain microbiological stability in liquid formulations. However, preservative-free systems are also available, e. g., the Advanced Preservative Free (APF) system from Aptar, which is vented, contains a filter membrane for air flow which prevents contamination, has a metal-free fluid path for oxidizing formulations, and can be used in any orientation. Additional nasal spray devices from Aptar and others are optimized with dispenser tips that prevent clogging (useful for high-viscosity and high-volatile formulations), actuators that do not need re-priming after long periods of disuse, etc. Additional nasal spray devices are propellant driven. Yet additional nasal spray devices include dry powder inhalers.

Traditional, simple single, bi-dose and multi-use metered dose spray pumps require priming and some degree of overfill to maintain dose conformity for the labeled number of doses. They are well suited for drugs to be administered daily over a prolonged duration, but due to the priming procedure and limited control of dosing, unless a specialty device is selected, they are less suited for drugs with a narrow therapeutic window of time in which to use the device, particularly if they are not used often. For expensive drugs and drugs intended for single administration or sporadic use and where tight control of the dose and formulation is of importance, single dose (UDS) or bi-dose spray (BDS) devices are preferred (see www.aptar.com). A simple variant of a single-dose spray device (MAD™) is offered by LMA (LMA, Salt Lake City, Utah, USA; (www.Imana.com). A nosepiece with a spray tip is fitted to a standard syringe. The liquid drug to be delivered is first drawn into the syringe and then the spray tip is fitted onto the syringe. This device has been used in academic studies to deliver, for example, a topical steroid in patients with chronic rhinosinusitis and in a vaccine study. A pre-filled device based on the same principle for one or two doses (Accuspray™, Becton Dickinson Technologies, Research Triangle Park, N. C., USA; (www.bd.com) is used to deliver the influenza vaccine FluMist™ (www.flumist.com), approved for both adults and children in the US market.

Pre-primed single dose and bi-dose devices are also available, and consist of a reservoir, a piston, and a swirl chamber (see, e. g., the UDS UnitDose™ and BDS BiDose™ devices from Aptar, formerly Pfeiffer). The spray is formed when the liquid is forced out through the swirl chamber. A pressure point mechanism incorporated in some devices secures reproducibility of the actuation force and emitted plume characteristics. Currently, marketed nasal migraine drugs like Imitrex® (sumatriptan) and Zomig® (zolmitriptan; Pfeiffer/Aptar single dose device), the marketed influenza vaccine FluMist (Becton Dickinson single dose spray device), and the intranasal formulation of naloxone for opioid overdose rescue, Narcan Nasal@(naloxone HCl, Adapt Pharma) are delivered with this type of device.

Devices recited herein may employ any of the pharmaceutical formulations and are useful in the methods disclosed herein. Accordingly, provided herein are devices adapted for oral spray delivery of a pharmaceutical formulation to a patient, comprising a reservoir with a therapeutically effective amount of epinephrine or epinephrine prodrug. Preferably, the oral spray is a sublingual spray.

In certain embodiments, the volume of the pharmaceutical formulation in the reservoir is not more than about 140 μL.

In certain embodiments, the volume of the pharmaceutical formulation in the reservoir is above about 125 μL and less than 140 μL.

In certain embodiments, about 100 μL of the pharmaceutical formulation in the reservoir is delivered to the patient in one actuation.

In certain embodiments, the spray device is filled with the pharmaceutical formulations using sterile filling.

### Single Dose Devices

In certain embodiments, the device is a single dose device, wherein the pharmaceutical formulation is present in one reservoir, and wherein the therapeutically effective amount of the epinephrine is delivered by one actuation of the device.

Also provided herein is a single use, pre-primed device adapted for oral spray delivery of a pharmaceutical formulation to a patient by one actuation of the device into the mouth of the patient under the tongue or on the tongue or into the buccal area having a single reservoir comprising about 100 μL of a pharmaceutical formulation as disclosed herein. In certain embodiments, the device is actuatable with one hand.

In certain embodiments, the delivery time is less than about 30 seconds. In certain embodiments, the delivery time is less than about 25 seconds. In certain embodiments, the delivery time is less than about 20 seconds. In certain embodiments, the delivery time is less than about 15 seconds.

### Bi-Dose Devices

In certain embodiments, said device is a bi-dose device, wherein a first volume is present in a first reservoir and a second volume of said formulation is present in a second reservoir, and wherein said therapeutically effective amount is delivered essentially by a first actuation of said device into one side of the sublingual space of said patient and a second actuation of said device into the other side of the sublingual space of said patient. In certain embodiments, both doses can be delivered to the same side of the sublingual space of said patient.

In certain embodiments, said first volume and said second volume combined is equal to not more than about 380 μL.

In certain embodiments, about 100 μL of said first volume of said formulation is delivered by said first actuation.

In certain embodiments, about 100 μL of said second volume of said formulation is delivered by said second actuation.

In certain embodiments, said bi-dose device is actuatable with one hand.

In certain embodiments, the delivery time is less than about 30 seconds. In certain embodiments, the delivery time is less than about 25 seconds. In certain embodiments. The delivery time is less than about 20 seconds. In certain embodiments, the delivery time is less than about 15 seconds.

Also provided are embodiments wherein any embodiment above may be combined with any one or more of these embodiments, provided the combination is not mutually exclusive.

This disclosure is further illustrated by the following examples, which are non-limiting.

### EXAMPLES

### Example 1. Representative Dipivefrin Formulations in Aqueous Vehicle for Clinical Use

TABLE 1.1

| | Aqueous F1 | Aqueous F2 | Aqueous F3 | Aqueous F4 | Aqueous F5 |
|---|---|---|---|---|---|
| Ingredient | (% w/v) | (% w/v) | (% w/v) | (% w/v) | (% w/v) |
| Dipivefrin HCl | 20.00 | 20 | 5 | 10 | 15 |
| EDTA disodium | 2.00 | 1.99 | 0.19 | 0.18 | 0.17 |
| Benzalkonium | 0.40 | 0.398 | 0.04645 | 0.044 | 0.0416 |
| chloride | | | | | |
| L-Menthol | 0.00 | 0 | 0.0475 | 0.045 | 0.0425 |
| Sucralose | 0.00 | 0.4 | 0.38 | 0.36 | 0.34 |
| 1N HCl | adjust pH | adjust pH | adjust pH | adjust pH | adjust pH |
| | to 3.08 | to 3.08 | to 2.91 | to 2.91 | to 2.91 |
| Purified water | q.s. to 100 | q.s. to 100 | q.s. to 100 | q.s. to 100 | q.s. to 100 |

### Example 2. Representative Dipivefrin Formulations in Non-Aqueous Vehicles for Clinical Use

TABLE 2.1

| | Non-Aq. | Non-Aq. | Non-Aq. | Non-Aq. | Non-Aq. |
|---|---|---|---|---|---|
| | Formulation | Formulation | Formulation | Formulation | Formulation |
| Ingredient | 1 (% w/v) | 2 (% w/v) | 3 (% w/v) | 4 (% w/v) | 5 (% w/v) |
| Dipivefrin HCl | 5.00 | 5 | 5 | 5 | 5 |
| L-Menthol | 2.50 | 5 | 2.38 | 0 | 0 |
| Eucalyptol | 0.00 | 0 | 0 | 0 | 0 |
| Eugenol | 0.00 | 0 | 8.08 | 0 | 8.5 |
| Sodium | 0.00 | 0 | 0 | 0 | 0 |
| deoxycholate | | | | | |
| Labrasol ® | 0.00 | 0 | 0.475 | 0 | 0 |
| Sucralose | 0.40 | 0 | 0.808 | 0 | 0 |
| Saccharin sodium | 0.00 | 0 | 0 | 0 | 0 |

TABLE 2.1

| | Non-Aq. | Non-Aq. | Non-Aq. | Non-Aq. | Non-Aq. |
|---|---|---|---|---|---|
| | Formulation | Formulation | Formulation | Formulation | Formulation |
| Ingredient | 1 (% w/v) | 2 (% w/v) | 3 (% w/v) | 4 (% w/v) | 5 (% w/v) |
| Propylene glycol | 50.00 | 50 | 38 | 50 | 50 |
| Dehydrated | q.s. to 100 | q.s. to 100 | q.s. to 100 | q.s. to 100 | q.s. to 100 |
| alcohol | | | | | |

TABLE 2.2

| | Non-Aq. | Non-Aq. | Non-Aq. | Non-Aq. | Non-Aq. |
|---|---|---|---|---|---|
| | Formulation | Formulation | Formulation | Formulation | Formulation |
| Ingredient | 6 (% w/v) | 7 (% w/v) | 8 (% w/v) | 9 (% w/v) | 10 (% w/v) |
| Dipivefrin | 5 | 5 | 5 | 5 | 5 |
| HCl | | | | | |
| L-Menthol | 2.5 | 0 | 0 | 1.25 | 0 |
| Eucalyptol | 0 | 0 | 0 | 0 | 2.5 |
| Eugenol | 0 | 0 | 0 | 0 | 0 |
| Sodium | 0 | 0 | 2.5 | 0 | 0 |
| deoxycholate | | | | | |
| Labrasol ® | 0 | 0.5 | 0 | 0 | 0 |
| Sucralose | 0 | 0 | 0 | 0 | 0 |
| saccharin | 0 | 0 | 0 | 0 | 0 |
| sodium | | | | | |
| Propylene | 50 | | 50 | 50 | 50 |
| glycol | | | | | |
| Dehydrated | q.s. to 100 | q.s. to 100 | q.s. to 100 | q.s. to 100 | q.s. to 100 |
| alcohol | | | | | |

TABLE 2.3

| | Non-Aq. | Non-Aq. | Non-Aq. | Non-Aq. | Non-Aq. |
|---|---|---|---|---|---|
| | Formulation | Formulation | Formulation | Formulation | Formulation |
| Ingredient | 11 (% w/v) | 12 (% w/v) | 13 (% w/v) | 14 (% w/v) | 15 (% w/v) |
| Dipivefrin | 10 | 5 | 5 | 5 | 5 |
| HCl | | | | | |
| L-Menthol | 5 | 2.5 | 2.5 | 5 | 2.565 |
| Eucalyptol | 0 | 0 | 0 | 0 | 0 |
| Eugenol | 0 | 0 | 0 | 0 | 0 |
| Sodium | 0 | 0 | 0 | 0 | 0 |
| deoxycholate | | | | | |
| Labrasol ® | 0 | 0 | 0 | 0 | 0 |
| Sucralose | 0 | 0.4 | 0 | 0.4 | 0.409 |
| saccharin | 0 | 0 | 1 | 0 | 0 |
| sodium | | | | | |

TABLE 2.3

| | Non-Aq. | Non-Aq. | Non-Aq. | Non-Aq. | Non-Aq. |
|---|---|---|---|---|---|
| | Formulation | Formulation | Formulation | Formulation | Formulation |
| Ingredient | 11 (% w/v) | 12 (% w/v) | 13 (% w/v) | 14 (% w/v) | 15 (% w/v) |
| Propylene | 50 | 50 | 50 | 50 | 51.3 |
| glycol | | | | | |
| Dehydrated | q.s. to 100 | q.s. to 100 | q.s. to 100 | q.s. to 100 | q.s. to 100 |
| alcohol | | | | | |

TABLE 2.4

| | Non-Aq. | Non-Aq. | Non-Aq. | Non-Aq. | Non-Aq. |
|---|---|---|---|---|---|
| | Formulation | Formulation | Formulation | Formulation | Formulation |
| Ingredient | 16 (% w/v) | 17 (% w/v) | 18 (% w/v) | 19 (% w/v) | 20 (% w/v) |
| Dipivefrin | 5 | 10 | 5 | 2.5 | 10 |
| HCl | | | | | |
| L-Menthol | 5 | 5 | 10 | 5.23 | 4.829 |
| Eucalyptol | 0 | 0 | 0 | 0 | 0 |
| Eugenol | 0 | 0 | 0 | 0 | 0 |
| Sodium | 0 | 0 | 0 | 0 | 0 |
| deoxycholate | | | | | |
| Labrasol ® | 0 | 0 | 0 | 0 | 0 |
| Sucralose | 0.4 | 0.4 | 0.4 | 0.417 | 0.385 |
| saccharin | 0 | 0 | 0 | 0 | 0 |
| sodium | | | | | |
| Propylene | 50 | 50 | 50 | 52.34 | 48.31 |
| glycol | | | | | |
| Dehydrated | q.s. to 100 | q.s. to 100 | q.s. to 100 | q.s. to 100 | q.s. to 100 |
| alcohol | | | | | |

### Example 3. Representative Dipivefrin Formulations in Aqueous/Organic Mixed Vehicles for Clinical Use

TABLE 3.1

| | Mixed | Mixed | Mixed | Mixed | Mixed |
|---|---|---|---|---|---|
| | Formulation | Formulation | Formulation | Formulation | Formulation |
| Ingredient | 1 (% w/v) | 2 (% w/v) | 3 (% w/v) | 4 (% w/v) | 5 (% w/v) |
| Dipivefrin HCl (mg) | 25 | 25 | 25 | 25 | 25 |
| Propylene | 0.5 | 0.4 | 0.25 | 0.1 | 0 |
| glycol/dehydrated alcohol | | | | | |
| (1; 1, v/v) (mL) | | | | | |
| Water (mL) | 0 | 0.1 | 0.25 | 0.4 | 0.5 |
| Total volume (mL) | 0.5 | 0.5 | 0.5 | 0.5 | 0.5 |

### Example 4. HPLC Methods for Assay, Purity and Chiral Analysis of Dipivefrin Hydrochloride Formulations

HPLC assay analysis was carried out on a Waters 2695 separations module equipped with an autosampler and a Waters 996 photodiode array detector. The HPLC parameters are as follows:
Column: Phenomenex Gemini C18, 100×4.6 mm, 3.0 μm
Flow rate: 1.0 mL/min
Detection: 260 nm
Temperature: Ambient
Injection volume: 10 μL
Run time: 10 min
Mobile phase: A: 0.1% TFA in water
   B: 0.1% TFA in acetonitrile

Gradient:

| Time (min) | Solvent A (%) | Solvent B (%) |
|---|---|---|
| 0 | 95 | 5 |
| 4 | 50 | 50 |
| 6 | 35 | 65 |
| 8 | 95 | 5 |
| 12 | 95 | 5 |

HPLC purity analysis was conducted on a Waters UPLC system equipped with a quaternary solvent manager (ACQ-QSM), Sample Manager (ACQ-FTN), photodiode array detector (ACQ-PDA) and QDa™ mass detector (ACQ-QDA). The UPLC method is described below:
Column: Acquity UPLC® BEH C18, 150×2.1 mm, 1.7 μm
Flow Rate: 0.4 mL/min
Detection: 260 nm
Column Temperature: 50° C.
Sample Temperature: 8° C.
Injection volume: 2 μL
Run time: 10 min
Mobile phase: A: H₂O:ACN:TFA=95:5:0.1 (v/v/v)
   B: H₂O:ACN:TFA=5:95:0.1 (v/v/v)

Gradient:

| Time (min) | Solvent A (%) | Solvent B (%) |
|---|---|---|
| 0 | 95 | 5 |
| 7 | 5 | 95 |
| 8 | 5 | 95 |
| 8.1 | 95 | 5 |
| 10 | 95 | 5 |

Chiral HPLC analysis was conducted on a Waters 2695 separations module equipped with an autosampler and a Waters 996 photodiode array detector. The HPLC method is described below:
Column: Chiralpak IH-3, 250×4.6 mm, 3.0 μm
Mobile phase/sample solvent: n-hexane:EtOH:TFA:Diethylamine=90:10:0.1:0.1 (v/v/v/v)
Flow rate: 1.0 mL/min
Detection: 260 nm
Column Temperature: Ambient
Injection volume: 20 μL
Run time: 10 min

### Example 5. Stability Data for Dipivefrin Hydrochloride Formulations

TABLE 5.1
Stability data of dipivefrin formulations in aqueous/organic mixed vehicles*

| | Initial | | 3 Days at 60° C. | | 8 Days at 60° C. | |
|---|---|---|---|---|---|---|
| | | 3- and 4- | | 3- and 4- | | 3- and 4- |
| | | monopivaloyl | | monopivaloyl | | monopivaloyl |
| Formulation | Appearance | epinephrine | Appearance | epinephrine | Appearance | Epinephrine |
| Mixed | clear | ND | clear | ND | clear | ND |
| Formulation 1 | colorless | | colorless | | colorless | |
| Mixed | clear | ND | clear | ND | light yellow | 0.96 |
| Formulation 2 | colorless | | colorless | | | |
| Mixed | clear | ND | slightly | 1.01 | Brown | 1.84 |
| Formulation 3 | colorless | | yellow | | | |
| Mixed | clear | ND | yellow | 1.23 | dark brown | 2.76 |
| Formulation 4 | colorless | | | | | |
| Mixed | clear | ND | yellow | 2.83 | dark brown | 6.43 |
| Formulation 5 | colorless | | | | | |

*HPLC UV detection at 260 nm

TABLE 5.2
Stability Data of Dipivefrin Formulations in Aqueous Vehicle

| | | | 3- and 4- | Dipivefrin pivalate |
|---|---|---|---|---|
| | Stability | Dipivefrin | Monopivaloylepinephrine | (RRT = 1.232) |
| Formulation | conditions | (% area) | IN-003/IN-004 (% area) | (% area) |
| Aqueous | Initial | 99.71 | 0.29 | 0 |
| Formulation 1 | 1 Month at 40° C. | 93.23 | 6.46 | 0.31 |
| | 66 days at 40° C. | 92.28 | 6.35 | 1.37 |
| Aqueous | Initial | 99.71 | 0.29 | 0 |
| Formulation 2 | 1 Month at 40° C. | 96.17 | 3.49 | 0.34 |
| | 66 days at 40° C. | 92.54 | 6.3 | 1.16 |
| Aqueous | Initial | 100 | 0 | 0 |
| Formulation 3 | 2 Months at 40° C. | 93.57 | 6.21 | 0.23 |
| Aqueous | Initial | 100 | 0 | 0 |
| Formulation 4 | 2 Months at 40° C. | 94.91 | 4.64 | 0.45 |
| Aqueous | Initial | 100 | 0 | 0 |
| Formulation 5 | 2 Months at 40° C. | 95.71 | 3.37 | 0.92 |

* HPLC UV detection at 260 nm

TABLE 5.3
Stability Data of Dipivefrin Formulation in Non-aqueous Formulation 1

| | | 42 Days | 73 Days | 3 Months |
|---|---|---|---|---|
| | Initial* | at 60° C. | at 60° C. | at 60° C. |
| Appearance | Clear | Clear | Clear | Clear |
| | colorless | colorless | colorless | colorless |
| | liquid | liquid | liquid | liquid |
| Assay | 102.4 | NT | NT | 104.3 |
| Purity (HPLC % area) | 100 | 99.39 | NT | 98.82 |
| Degradants (HPLC % area) | | | | |
| 3- and 4-Monopivaloyepinephrine | ND | 0.37 | NT | 0.54 |
| (IN-003/IN-004) | | | | |
| Pivaloyl amide (IN-011) | ND | 0.24 | NT | 0.64 |
| (RRT = 1.422) | | | | |
| Dipivefrin pivalate (IN-010) | ND | ND | NT | ND |
| (RRT = 1.232) | | | | |
| Chiral impurity (IN-006) | 0.35 | NT | 0.19 | 0.36 |

*Obtained from a freshly prepared identical formulation; ND: Not detected; NT: Not Tested

Dipivefrin in non-aqueous vehicles remains stable for at least 3 months when stored at 60° C.

### Example 6. Clinical Studies of Dipivefrin Formulations

The following clinical protocols were conducted, or may be carried out, in healthy adult human volunteers under fasting conditions to assess the safety/tolerability, optimal dosing, and the rate and extent of epinephrine absorption after intraoral (sublingual or lingual) administration of dipivefrin formulations using an oral liquid spray device or via exact pipetting. As a comparator, epinephrine pharmacokinetics was also assessed following intramuscular injection of 0.3 mg epinephrine delivered by auto injector EpiPen®.

Healthy males and non-pregnant, non-lactating females aged 18-45 years within the BMI range of 18.50-29.90 kg/m², inclusive, were selected according to routine physical examination, medical history, vital signs, ECG, and normal plasma cholinesterase activity along with other clinical laboratory inclusion and exclusion criteria.

The subjects were screened within 28 days prior to start of dosing. The screening was carried out after obtaining a signed informed consent for screening or study-specific informed consent from subjects.

### Clinical Study 1

Three adult human subjects were enrolled in this study. The study was a Single Ascending Dose (SAD) study with 4 doses of dipivefrin formulation in aqueous vehicle administered (Table 6.1). There was a minimum 7-day washout period between all treatment periods following each administration of study drug.

TABLE 6.1
Summary of Doses Used for Clinical Study

| | | | Drug | Dose | |
|---|---|---|---|---|---|
| Treatment | Administration | Dosing | concentration | volume | Dose* |
| Period | site | Device | (mg/mL) | (μl) | (mg) |
| I | Top of the | Sprayer | 50 | 100 | 5.0 |
| | tongue | | | | |
| II | Top of the | Sprayer | 150 | 100 | 15.0 |
| | tongue | | | | |
| III | Under the | Pipette | 100 | 50 | 5.0 |
| | tongue | | | | |
| IV | Under the | Sprayer | 100 | 50 | 5.0 |
| | tongue | | | | |

*Dose denoted as HCl salt.

Drug compounding. A vehicle, components listed in Table 6.2, is prepared according to the following protocol.

TABLE 6.2

| Component Name | Quantity | |
|---|---|---|
| Edetate (EDTA) Disodium USP | 200 | mg ⁽¹⁾ |
| Sucralose granular NF | 400 | mg ⁽¹⁾ |
| L-Menthol USP | 50 | mg ⁽¹⁾ |
| Benzalkonium Chloride Solution NF | 100 | mg ⁽¹⁾ |
| 1N Hydrochloric acid USP | 0.400 | mL |

| | |
|---|---|
| Sterile Water for Injection, USP | Q.S. 100 mL |

⁽¹⁾ Allowance for weight +/− 0.5 mg for EDTA, Sucralose, L-Menthol, and Benzalkonium Chloride

Preparation of Dosing Vehicle. 200 mg of Edetate Disodium USP, 400 mg of Sucralose NF and 50 mg of L-menthol USP are placed in a clean 100-mL volumetric flask.

Weigh 100 mg of Benzalkonium Chloride 50% solution NF are weighed into a clean 2-mL glass vial, 1 ml of water is added to the glass vial and the contents of the vial are transferred to the 100-mL volumetric flask. Additional rinses of the glass vial (1 ml each) are performed to complete transfer of benzalkonium chloride into the volumetric flask. About 85 mL of Water for Injection is added into the volumetric flask. The contents of the volumetric flask are sonicated at 40-50° C. until full dissolution of all solid materials is complete. The volumetric flask is cooled to room temperature and 0.400 mL of 1N HCl USP is added to the volumetric flask. Additional water is added to the volumetric flask to bring the solution to the desired volume. The volumetric flask is inverted 3-4 times to mix well. The resulting vehicle solution should be clear, colorless, and free of visible particles. The pH of the vehicle should be around 3 (range 2.5-3.5).

Stability Assessment of the Vehicle. Due to the presence of benzalkonium chloride as a preservative at 0.05% w/w level, a concentration comparable to or higher than those found in approved nasal spray and ophthalmic products, this vehicle is considered well preserved for microbial stability. The chemical stability of the vehicle is assessed by appearance and pH. There is no change from initial in appearance and pH after 40 days of storage at ambient temperature indicating that the vehicle is stable for 40 days at ambient temperature.

Study drug compounding. Table 6.2A provides the drug amounts used in this study.

TABLE 6.2A
Compositions of study drug for treatment periods I-IV

| Treatment Period | I | II | III | IV |
|---|---|---|---|---|
| L-Dipivefrin HCl GMP grade (mg) | 250.00 | 750.00 | 500.00 | 500.00 |
| Dose Vehicle | q.s. to 5 mL | q.s. to 5 mL | q.s. to 5 mL | q.s. to 5 mL |

The test solution should be clear, colorless, and free of visible particles. It is stable at both ambient and refrigerated temperature for 72 hours after preparation based on stability data shown in Table 6.3 below.

TABLE 6.3
72-Hour Stability Results of L-dipivefrin HCl sublingual
solution at 20 mg/mL and 150 mg/mL concentrations under
both refrigerated and standard stability conditions

| | Storage: 2° C.-8° C. | | | 25° C./60% RH | | |
|---|---|---|---|---|---|---|
| | API peak | API peak | | API peak | API peak | |
| | area at | area at | % | area at | Area at | % |
| Formulation | Initial | 72 hours | Initial | Initial | 72 hours | Initial |
| 20 mg/mL | 2005.7 | 2034.9 | 101.5 | 2005.7 | 2017.6 | 100.6 |
| 150 mg/mL | 2065.2 | 2046.1 | 99.1 | 2065.2 | 2044.1 | 99.0 |

Study drug administration. In all treatment periods, after an overnight fast of at least 10 hours, all subjects received a single oral dose of L-Dipivefrin Oral Liquid Spray/Solution under fasted conditions administered via an oral liquid spray device or via exact pipetting.

PK Sampling Schedule. A total of eighteen (18) blood samples of 1×6 mL were collected in chilled pre-labelled vacutainer tubes containing K2EDTA during each period. The tubes were inverted several times to mix and were kept on wet ice immediately after blood collection until centrifugation within 60 minutes of collection.

Two pre-dose (−30 minutes and 0 minutes) blood samples of 6 mL each were collected within 30 minutes before dosing in each period. The −30 minute sample should be collected as close as possible to the scheduled time, within 30 minutes prior to dosing. The 0 minute sample should be collected within 5 minutes of the scheduled dosing time. The post-dose blood samples of 6 mL each were collected at 2.00, 4.00, 6.00, 8.00, 10.00, 12.00, 15.00, 20.00, 30.00, 45.00, 60.00, 90.00, 120.00, 180.00, 240.00, and 360.00 minutes in each period.

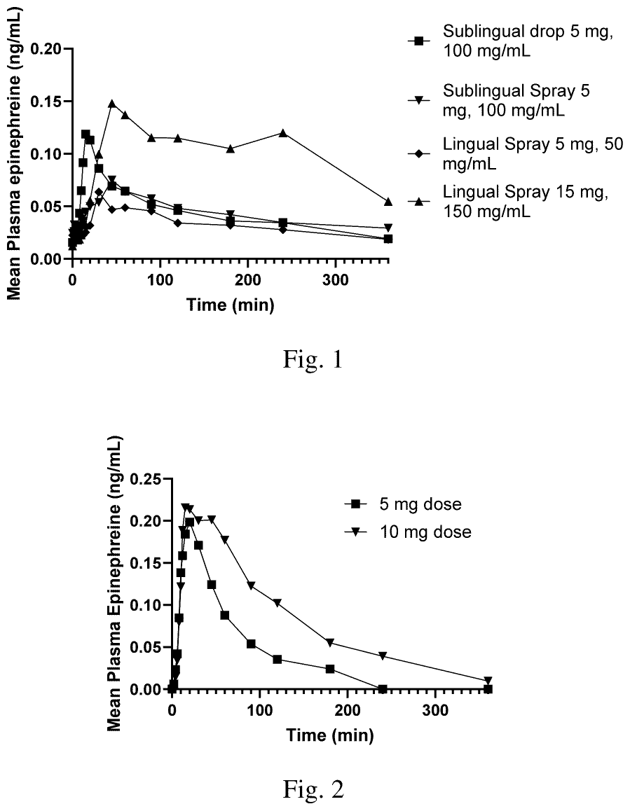

PK Analysis. Mean Plasma Epinephrine was estimated in plasma using a validated LC/MS/MS method. The Epinephrine Mean Concentration-Time profiles From Study 1 are shown in FIG. 1. PK parameters from study 1 are listed in Table 6.4 below.

TABLE 6.4
Epinephrine PK Parameters Following a Single Dose
of L-dipivefrin HCl From Each Study Period

| Treatment | Cₘₐₓ (ng/mL) | AUC₀₋ₗₐₛₜ (min · | Median |
|---|---|---|---|
| Period | (CV %) | ng/mL) (CV %) | Tₘₐₓ (min) |
| I | 0.0681 (33.4) | 12.15 (30.3) | 30 |
| II | 0.158 (5.5) | 36.79 (4.9) | 60 |
| III | 0.122 (72.7) | 15.99 (35.9) | 15 |
| IV | 0.0751 (28.5) | 16.11 (42.1) | 45 |

Safety. All treatments in this study were safe and well-tolerated. All treatment emergent adverse events (TEAEs) were either mild or moderate in severity and were transient and self-resolved without intervention. There were no serious adverse events (SAEs).

### Example 7. Single Ascending Dose Clinical Study (Study 2)

Six subjects were enrolled in this study. The study was a Single Ascending Dose (SAD) study with 2 doses of dipivefrin formulation in a non-aqueous vehicle administered (Table 7.1). There were a minimum 7-day washout period between the treatments.

TABLE 7.1
Summary of Clinical Study 2

| | | | Drug | Dose | |
|---|---|---|---|---|---|
| Treatment | Administration | | concentration | volume | Dose* |
| Period | site | Dosing Device | (mg/mL) | (μl) | (mg) |
| I | Under the tongue | Sprayer | 50 | 100 | 5.0 |
| II | Under the tongue | Sprayer | 100 | 100 | 10.0 |

*Study dose denoted as HCl salt

Study drug compounding. Table 7.2 provides the amounts for excipients used in the vehicle this study.

TABLE 7.2
Vehicle Component Amounts

| Component Name | Quantity | |
|---|---|---|
| L-Menthol - USP | 1.34 | g⁽¹⁾ |
| Sucralose granular NF | 0.107 | g⁽²⁾ |
| Propylene Glycol - USP | 13.42 | g⁽³⁾ |

| | |
|---|---|
| Dehydrated Alcohol - USP | q.s. to 25 mL |

⁽¹⁾Allowance for weight:
⁽¹⁾+/−0.01 g (1.33-1.35 g),
⁽²⁾weight +/−0.0005 g (0.1065-0.1075 g),
⁽³⁾+/− 0.01 g (13.41-13.43 g)

Preparation of Dosing Vehicle. Place 1.34 g of L-Menthol USP and 0.107 g of Sucralose NF in a dry clean 25-mL volumetric flask. Add 13.42 g of Propylene Glycol USP and then 10 mL of Dehydrated Alcohol into the same 25-mL volumetric flask. Vortex to allow the contents in the volumetric flask to mix. Sonicate the contents of the volumetric flask at ambient until full dissolution of all solid materials. Add additional Dehydrated Alcohol to the volumetric flask to volume. Invert the volumetric flask 3-4 times to mix well. The vehicle solution should be clear, colorless, and free of visible particles. Store the Dosing Vehicle at ambient temperature.

Table 7.3. provides the compositions of study drug for treatment periods I and II.

TABLE 7.3

| Treatment Period | I | II |
|---|---|---|
| L-Dipivefrin HCl GMP grade (mg) | 250.00 | 500.00 |
| Dose Vehicle | Q.S. to 5 mL | Q.S. to 5 mL |

The test solution is clear, colorless, and free of visible particles. It is stable at both ambient and refrigerated temperature for at least 10 days after preparation based on stability data shown in Tables 7.4 and 7.5 below.

TABLE 7.4
10-day Stability Results of L-dipivefrin HCl sublingual solution at both 25
mg/mL and 100 mg/mL concentrations under refrigerated storage conditions.

| | Initial | 72 Hours | | | 10 days | | |
|---|---|---|---|---|---|---|---|
| | API peak | API peak | % | | API peak | % | |
| Formulation | area | Area | Initial | Appearance | Area | Initial | Appearance |
| 25 mg/mL | 161622 | 171679 | 106.2 | Clear solution, | 162453 | 100.5 | Clear solution, |
| | | | | no change | | | no change |
| | | | | from initial | | | from initial |
| 100 mg/mL | 645181 | 649326 | 100.6 | Clear solution, | 651414 | 101.0 | Clear solution, |
| | | | | no change | | | no change |
| | | | | from initial | | | from initial |

TABLE 7.5
10-Day Stability Results of L-dipivefrin HCl sublingual solution at both
25 mg/mL and 100 mg/mL concentrations under ambient storage conditions

| | Initial | 72 Hours | | | 10 days | | |
|---|---|---|---|---|---|---|---|
| | API peak | API peak | % | | API peak | % | |
| Formulation | area | Area | Initial | Appearance | Area | Initial | Appearance |
| 25 mg/mL | 161622 | 167748 | 103.8 | Clear solution, | 158455 | 98.0 | Clear solution, |
| | | | | no change | | | no change |
| | | | | from initial | | | from initial |
| 100 mg/mL | 645181 | 646023 | 100.1 | Clear solution, | 642973 | 99.7 | Clear solution, |
| | | | | no change | | | no change |
| | | | | from initial | | | from initial |

Study drug administration. In both treatment periods, after an overnight fast of at least 10 hours, all subjects received a single oral dose of L-Dipivefrin Oral Liquid Spray/Solution under fasted conditions administered via an oral liquid spray device under the tongue.

PK Sampling Schedule. A total of eighteen (18) blood samples of 1×6 mL were collected in chilled pre-labelled vacutainer tubes containing K₂EDTA during each period. The tubes were inverted several times to mix and were kept on wet ice immediately after blood collection until centrifugation within 60 minutes of collection.

Two pre-dose (−30 minutes and 0 minutes) blood samples of 6 mL each are collected within 30 minutes before dosing in each period. The −30 minute sample is collected as close as possible to the scheduled time, within 30 minutes prior to dosing. The 0 minute sample is collected within 5 minutes of the scheduled dosing time. Post-dose blood samples of 6 mL each are collected at 2.00, 4.00, 6.00, 8.00, 10.00, 12.00, 15.00, 20.00, 30.00, 45.00, 60.00, 90.00, 120.00, 180.00, 240.00, and 360.00 minutes in each period.

PK Analysis. Epinephrine was estimated in plasma using a validated LC/MS/MS method. The Epinephrine Mean Concentration-Time profiles from this study are shown in FIG. 2. The PK parameters from this study are listed in Table 7.6, below.

TABLE 7.6
Epinephrine PK Parameters Following a Single Dose
of L-Dipivefrin HCl from each study Period

| Treatment | Cₘₐₓ (ng/mL) | AUC₀₋ₗₐₛₜ (min · | Median |
|---|---|---|---|
| Period | (CV %) | ng/mL) (CV %) | Tₘₐₓ (min) |
| I | 0.216 (106) | 12.8 (86) | 26 |
| II | 0.302 (55) | 27.1 (28) | 29 |

Safety. All treatments in this study were safe and well-tolerated. Treatment emergent adverse events (TEAEs) were all mild in severity and were transient and self-resolved without intervention. There were no serious adverse events (SAEs).

### Example 8. Comparative Study, IM Administration of Epinephrine (Study 3)

Five subjects were enrolled in this study. All subjects received a single dose of 0.3 mg intramuscular epinephrine delivered by EpiPen®.

Study drug administration. After an overnight fast of at least 10 hours, all subjects received an intramuscular injection of Epipen® (Epinephrine Injection) Auto-Injector 0.3 mg/0.3 mL into the anterolateral aspect of the subject's thigh.

PK Sampling Schedule. A total of eighteen (18) blood samples of 1×6 mL were collected in chilled pre-labelled vacutainer tubes containing K2EDTA during each period. The tubes were inverted several times to mix and were kept on wet ice immediately after blood collection until centrifugation within 60 minutes of collection.

Two pre-dose (−30 minutes and 0 minutes) blood samples of 6 mL each were collected within 30 minutes before dosing in each period. The −30 minute sample should be collected as close as possible to the scheduled time, within 30 minutes prior to dosing. The 0 minute sample should be collected within 5 minutes of the scheduled dosing time.

The post-dose blood samples of 6 mL each were collected at 2.00, 4.00, 6.00, 8.00, 10.00, 12.00, 15.00, 20.00, 30.00, 45.00, 60.00, 90.00, 120.00, 180.00, 240.00, and 360.00 minutes in each period.

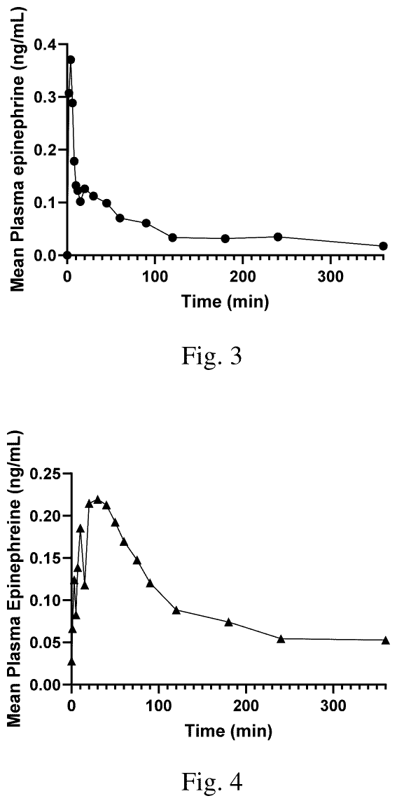

PK Analysis. Epinephrine was estimated in plasma using a validated LC/MS/MS method. The Epinephrine Mean Concentration-Time profiles are shown in FIG. 3 PK parameters from this study are listed in Table 8.1 below.

TABLE 8.0
Epinephrine PK Parameters Following
a Single Dose of IM Epinephrine

| Cₘₐₓ (ng/mL) | AUC₀₋ₗₐₛₜ (min · | Median |
|---|---|---|
| (CV %) | ng/mL) (CV %) | Tₘₐₓ (min) |
| 0.508 (68.2) | 18.1 (33.7) | 4 |

### Example 9. Comparative Study of IM Administration of Epinephrine (Study 4)

Eight subjects were included in this study. All subjects received a single dose of 0.3 mg intramuscular epinephrine delivered by EpiPen®.

Study drug administration. After an overnight fast of at least 10 hours, all subjects received an intramuscular injection of Epipen® (Epinephrine Injection) Auto-Injector 0.3 mg/0.3 mL into the anterolateral aspect of the subject's thigh.

PK Sampling Schedule. A total of eighteen (18) blood samples of 1×6 mL were collected in chilled pre-labelled vacutainer tubes containing K2EDTA during each period. The tubes were inverted several times to mix and were kept on wet ice immediately after blood collection until centrifugation within 60 minutes of collection.

Two pre-dose (−30 minutes and 0 minutes) blood samples of 6 mL each were collected within 30 minutes before dosing in each period. The −30 minute sample is collected as close as possible to the scheduled time, within 30 minutes prior to dosing. The 0 minute sample is collected within 5 minutes of the scheduled dosing time.

The post-dose blood samples of 6 mL each were collected at 1, 3, 5, 7, 10, 15, 20, 30, 40, 50 min and 1.00, 1.25, 1.50, 2.00, 3.00, 4.00 and 6.00 hours.

PK Analysis. The Epinephrine Mean Concentration-Time profiles from this study are shown in FIG. 4

PK Analysis. PK parameters from this study are listed in Table 9.1 below.

TABLE 9.1

| Cₘₐₓ (ng/mL) | AUC₀₋ₗₐₛₜ (min · | Median |
|---|---|---|
| (CV %) | ng/mL) (CV %) | Tₘₐₓ (min) |
| 0.328 (31.4) | 35.9 (19.5) | 30 |

### Example 10. Epinephrine Pk Comparison Between L-Dipivefrin HCl Formulations and EpiPen 0.3 mg

FIG. 5 presents a comparison between the epinephrine mean concentration-Time profiles from Clinical Ex. 7 and Epipen Examples 8 and 9.

TABLE 10.1
Epinephrine PK Parameters Following a Single Dose of L-dipivefrin
HCl (Example 7) and Epipen 0.3 mg (Exs. 9 & 10)

| | Cₘₐₓ (ng/mL) | AUC₀₋ₗₐₛₜ (min · | Median |
|---|---|---|---|
| Treatment | (CV %) | ng/mL) (CV %) | Tₘₐₓ (min) |
| Study 2, Period I | 0.216 (105.6) | 16.2 (67.4) | 26 |
| Study 2, Period II | 0.302 (55.3) | 29.6 (26.7) | 29 |
| Study 3, Epipen 0.3 mg | 0.508 (68.2) | 18.1 (33.7) | 4 |
| Study 4, EpiPen 0.3 mg | 0.328 (31.4) | 35.9 (19.5) | 30 |

As shown in Table 10.1, L-dipivefrin HCl oral formulations administered sublingually demonstrate comparable epinephrine exposures compared to Epipen 0.3 mg, the standard of care.

### Example 11. Pharmacodynamics (PD) Response Comparison Between L-Dipivefrin HCl Formulation 10 mg Dose and Epipen 0.3 mg

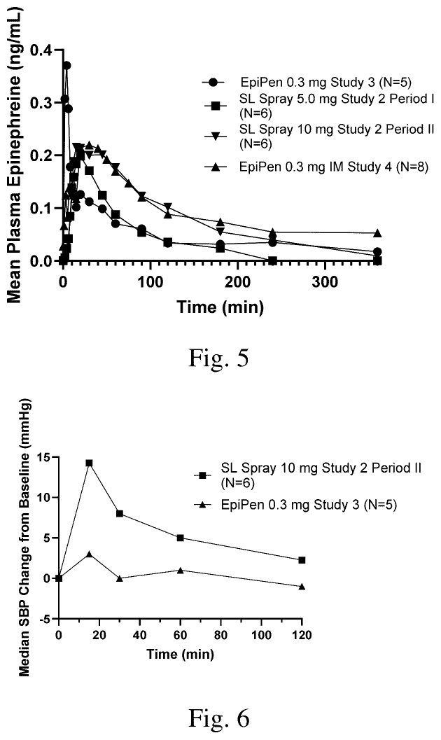

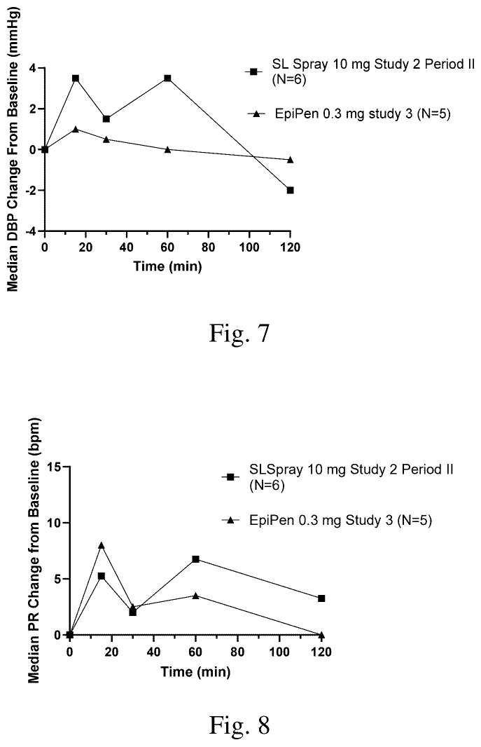

FIGS. 6-8 present comparisons between the median PD Responses (SBP, DBP and PR change from baseline)-Time profiles from Clinical Ex. 7 and Epipen Example 8. As shown in the Figures, sublingual dipivefrin HCl liquid dosage form containing 10 mg dipivefrin HCl provides a comparable or greater PD response compared to EpiPen 0.3 mg injection two hours postdose.

1. A non-aqueous liquid epinephrine or epinephrine prodrug formulation, comprising (i) epinephrine or an epinephrine prodrug as a pharmaceutical agent, and (ii) a nonaqueous solvent, wherein the formulation is stable for at least 3 months at temperatures from −20° C. to 60° C.

2. A non-aqueous liquid epinephrine or epinephrine prodrug formulation comprising (i) epinephrine or an epinephrine prodrug as a pharmaceutical agent, (ii) at least 30% (w/v) propylene glycol, polyethylene glycol, or glycerol, or a combination thereof, (iii) at least 10% dehydrated ethyl alcohol, a terpene or terpenoid, or a combination thereof, and optionally a surfactant.

3. The formulation of claim 1, wherein the pharmaceutical agent is dipivefrin hydrochloride.

4. The formulation of claim 2, additional comprising 0.1%-25% (w/v) of a terpene or a terpenoid selected from L-Menthol, Eucalyptol, and Eugenol, and the combinations thereof.

5. (canceled)

6. The formulation of claim 4, wherein the formulation comprises a surfactant selected from sodium deoxycholate, a caprylocaproyl macrogol-8 glyceride, a caprylocaproyl polyoxyl-8 glyceride, a polyoxylglyceride, a polyoxyethylene stearate, a sorbitan fatty acid ester, a poloxamer, and a combination of any of the foregoing.

7. The formulation of claim 6, additionally comprising a sweetener.

8. The formulation of claim 2, wherein (i) the pharmaceutical agent is L-dipivefrin hydrochloride, the formulation comprises (ii) at least 30% propylene glycol (w/v), (iii) at least 10% (w/v) dehydrated ethyl alcohol, (iii) 0.001 to 0.50 g/mL of a terpene selected from L-Menthol, Eucalyptol, and Eugenol, and optionally 0.1 to 5% (w/v) of a surfactant.

9. (canceled)

10. The formulation of claim 8, wherein the surfactant comprises a caprylocaproyl macrogol-8 glyceride, a caprylocaproyl polyoxyl-8 glyceride, a polyoxylglyceride, or a combination of the foregoing.

11. The formulation of claim 8, additional comprising 0.1 to 2.5% of a sweetener.

12-13. (canceled)

14. The formulation of claim 8, wherein the formulation comprises (i) 10 mg/mL to 300 mg/mL dipivefrin HCl, (ii) 30% to 70% (w/v) propylene glycol, polyethylene glycol, or glycerol, or a combination thereof, and (iii) 40% to 70% (w/v) dehydrated ethyl alcohol, and (iv) 0.001 to 0.50 g/mL (w/v) of a terpene selected from L-Menthol, Eucalyptol, and Eugenol.

15. (canceled)

16. The formulation of claim 1, comprising (i) 5% to 20% (w/v) L-dipivefrin hydrochloride, (ii) 45% to 60% (w/v) propylene glycol, polyethylene glycol, or glycerol, or a combination thereof, (iii) 30% to 50% (w/v) dehydrated ethyl alcohol, and (iv) 0.001 to 0.50 g/mL (w/v) of a terpene selected from L-Menthol, Eucalyptol, and Eugenol.

17. A unit dosage form, comprising the formulation of claim 16, comprising either 10 mg dipivefrin hydrochloride or 15 mg dipivefrin hydrochloride based on the weight of the hydrochloride salt or comprising a either 10 mg dipivefrin hydrochloride or 15 mL dipivefrin hydrochloride based on the weight of the free base (i.e. either 11.03 mL dipivefrin hydrochloride or 16.54 mL dipivefrin hydrochloride).

18. (canceled)

19. The formulation of claim 16, additionally comprising 0.1% to 1.0% (w/v) of a sweetener, wherein the terpene is L-menthol, and optionally comprising 0.5% to 52.0% of a non-ionic surfactant (e.g. Caprylocaproyl Polyoxyl-8-glycerides).

20. A method for systemic delivery of a therapeutically effective amount of epinephrine to a subject, comprising orally administering a formulation of claim 3 to the subject.

21. A method for treating a condition responsive to epinephrine in a subject comprising orally administering a therapeutically effective amount of a formulation of claim 3 to the subject.

22. The method of claim 21, wherein the formulation or unit dosage form is administered as an oral spray, oral film, liquid-filled capsule, or buccal, lingual, or sublingual drop formulation.

23-24. (canceled)

25. The method of claim 21, wherein the condition is a breathing difficulty or a Type I allergic reaction.

26. (canceled)

27. The method of claim 25, wherein the breathing difficulty is asthma, bronchitis, emphysema, croup, COPD, or a respiratory infection and the Type I allergic reaction symptoms include anaphylaxis and/or urticaria.

28-30. (canceled)

31. The method of claim 20, wherein the formulation is administered as a dosage form comprising 0.01 mg to 150 mg, 0.01 mg to 100 mg, 0.01 mg to 50 mg, 0.1 mg to 20 mg, 0.1 mg to 10 mg, 0.1 mg to 5 mg, 0.1 mg to 3 mg, 2.5 mg, 2 mg, or 1.5 mg of the dipivefrin hydrochloride.

32. (canceled)

33. The method of claim 20, wherein the formulation is administered in an amount sufficient to provide an epinephrine plasma Cₘₐₓ of 0.1 to 50.0 ng/mL in the subject.

34. The method of claim 27, wherein the formulation is administered in an amount sufficient to provide pharmacodynamic responses (changes in SBP, DPB and PR) comparable to or greater than the pharmacodynamic responses of an US FDA-approved injectable dosage form comprising epinephrine, when the US FDA-approved injectable dosage form is administered either intramuscularly or subcutaneously.

35. The method of claim 34, wherein the US FDA-approved dosage form is an injectable epinephrine dosage form comprising 0.5 mg epinephrine, 0.3 mg epinephrine, 0.15 mg epinephrine, or 0.1 mg epinephrine and is administered intramuscularly.

36. (canceled)

37. The method of claim 20, wherein the formulation comprises L-dipivefrin hydrochloride as the pharmaceutical agent and the method provides a therapeutically effective amount of epinephrine within 30 minutes of administration, within 15 minutes of administration, within 10 minutes of administration, or within 5 minutes of administration.

38-42. (canceled)
